(19) United States
(12) Patent Application Publication
(10) Pub. No.: US 2022/0267777 A1
(43) Pub. Date: Aug. 25, 2022

(54) DIFFERENTIAL KNOCKOUT OF A HETEROZYGOUS ALLELE OF RPE65

(71) Applicants: David Baram, Tel Aviv (IL); Lior Izhar, Tel Aviv (IL); Asael Herman, Ness-Ziona (IL); Rafi Emmanuel, Ramla (IL); Michal Golan Mashiach, Ness-Ziona (IL); Joseph Georgeson, Rehovot (IL)

(72) Inventors: David Baram, Tel Aviv (IL); Lior Izhar, Tel Aviv (IL); Asael Herman, Ness-Ziona (IL); Rafi Emmanuel, Ramla (IL); Michal Golan Mashiach, Ness-Ziona (IL); Joseph Georgeson, Rehovot (IL)

(73) Assignee: EmendoBio Inc., Wilmington, DE (US)

(21) Appl. No.: 17/625,290

(22) PCT Filed: Jul. 10, 2020
(86) PCT No.: PCT/US2020/041569

§ 371 Date: Jan. 6, 2022

Related U.S. Application Data
(60) Provisional application No. 62/872,514, filed on Jul. 10, 2019.

(51) Int. Cl. C12N 15/113

(52) U.S. Cl. CPC C12N 15/1137; C12N 2320/34; C12N 2310/20; C12Y 301/01064

(57) ABSTRACT

RNA molecules comprising a guide sequence portion having 17-25 contiguous nucleotides in the sequence set forth in any one of SEQ ID NOs: 1-49516 and compositions, methods, and uses thereof.

This application claims the benefit of U.S. Provisional Application No. 62/872,514, filed Jul. 10, 2019, the contents of which are hereby incorporated by reference.

Throughout this application, various publications are referenced, including referenced in parenthesis. The disclosures of all publications mentioned in this application in their entireties are hereby incorporated by reference into this application in order to provide additional description of the art to which this invention pertains and of the features in the art which can be employed with this invention.

### REFERENCE TO SEQUENCE LISTING

This application incorporates-by-reference nucleotide sequences which are present in the file named “200710_91034-A-PCT_Sequence_Listing_AWG.txt”, which is 9,361 kilobytes in size, and which was created on Jul. 6, 2020 in the IBM-PC machine format, having an operating system compatibility with MS-Windows, which is contained in the text file filed Jul. 10, 2020 as part of this application.

### BACKGROUND OF INVENTION

There are several classes of DNA variation in the human genome, including insertions and deletions, differences in the copy number of repeated sequences, and single nucleotide polymorphisms (SNPs). A SNP is a DNA sequence variation occurring when a single nucleotide (adenine (A), thymine (T), cytosine (C), or guanine (G)) in the genome differs between human subjects or paired chromosomes in an individual. Over the years, the different types of DNA variations have been the focus of the research community either as markers in studies to pinpoint traits or disease causation or as potential causes of genetic disorders.

A genetic disorder is caused by one or more abnormalities in the genome. Genetic disorders may be regarded as either “dominant” or “recessive.” Recessive genetic disorders are those which require two copies (i.e., two alleles) of the abnormal/defective gene to be present. In contrast, a dominant genetic disorder involves a gene or genes which exhibit(s) dominance over a normal (functional/healthy) gene or genes. As such, in dominant genetic disorders only a single copy (i.e., allele) of an abnormal gene is required to cause or contribute to the symptoms of a particular genetic disorder. Such mutations include, for example, gain-of-function mutations in which the altered gene product possesses a new molecular function or a new pattern of gene expression. Other examples include dominant negative mutations, which have a gene product that acts antagonistically to the wild-type allele.

### Dominant RPE65 Mutation Related Disorder

Most of the mutations in the retinal pigment epithelium-specific 65 kDa protein gene (RPE65) are recessive. However, an Asp477Gly in Exon 13 was shown to be a dominant RPE65 mutation resulting in retinitis pigmentosa (Sara J. Bowne et al. 2011). Yet another mutation originally characterized to be semidominant in mice (Wright et al. 2013) was identified in humans as well (R44X_rs368088025_G>A).

### SUMMARY OF THE INVENTION

Disclosed is an approach for knocking out the expression of a dominant-mutated RPE65 allele by disrupting the dominant-mutated allele or degrading the resulting mRNA.

The present disclosure provides a method for utilizing at least one naturally occurring nucleotide difference or polymorphism (e.g., single nucleotide polymorphism (SNP)) for distinguishing/discriminating between two alleles of a gene, one allele bearing a mutation such that it encodes a mutated protein causing a disease phenotype (“mutated allele”) and a particular sequence in a SNP position (REF/SNP), and the other allele encoding for a functional protein (“functional allele”). In some embodiments, the SNP position is utilized for distinguishing/discriminating between two alleles of a gene bearing one or more disease associated mutations, such as to target one of the alleles bearing both the particular sequence in the SNP position (SNP/REF) and a disease associated mutation. In some embodiments, the disease-associated mutation is targeted. In some embodiments, the method further comprises the step of knocking out expression of the mutated protein and allowing expression of the functional protein.

The present disclosure also provides a method for modifying in a cell a mutant allele of the retinal pigment epithelium-specific 65 kDa protein gene (RPE65) gene having a mutation associated with a dominant RPE65 gene disorder, the method comprising
introducing to the cell a composition comprising:
   a CRISPR nuclease or a sequence encoding the CRISPR nuclease; and
   a first RNA molecule comprising a guide sequence portion having 17-25 nucleotides or a nucleotide sequence encoding the same,
wherein a complex of the CRISPR nuclease and the first RNA molecule affects a double strand break in the mutant allele of the RPE65 gene.

According to embodiments of the present invention, there is provided a first RNA molecule comprising a guide sequence portion having 17-25 contiguous nucleotides containing nucleotides in the sequence set forth in any one of SEQ ID Nos: 1-49516.

According to some embodiments of the present invention, there is provided a composition comprising an RNA molecule comprising a guide sequence portion having 17-25 contiguous nucleotides containing nucleotides in the sequence set forth in any one of SEQ ID NOs: 1-49516 and a CRISPR nuclease.

According to some embodiments of the present invention, there is provided a method for inactivating a mutant RPE65 allele in a cell, the method comprising delivering to the cell a composition comprising an RNA molecule comprising a guide sequence portion having 17-25 contiguous nucleotides containing nucleotides in the sequence set forth in any one of SEQ ID NOs: 1-49516 and a CRISPR nuclease. In some embodiments, the cell is a stem cell. In some embodiments, the stem cell is an autologous pluripotent stem cell or an induced pluripotent stem cell (iPSC). In some embodiments, the stem cell is differentiated into a retinal pigment epithelium cell. In some embodiments, the cell is a retinal pigment epithelium cell. In some embodiments, the delivering to the cell is performed in vitro, ex vivo, or in vivo. In some embodiments, the method is performed ex-vivo and the cell is provided/explanted from an individual patient. In some embodiments, the method further comprises the step of introducing the resulting cell, with the modified/knocked out mutant RPE65 allele, into the individual patient (e.g. autologous transplantation).

According to some embodiments of the present invention, there is provided a method for treating a dominant RPE65 gene disorder, the method comprising delivering to a cell of a subject having a dominant RPE65 gene disorder a composition comprising an RNA molecule comprising a guide sequence portion having 17-25 contiguous nucleotides containing nucleotides in the sequence set forth in any one of SEQ ID NOs: 1-49516 and a CRISPR nuclease.

According to some embodiments of the present invention, there is provided use of a composition comprising an RNA molecule comprising a guide sequence portion having 17-25 contiguous nucleotides containing nucleotides in the sequence set forth in any one of SEQ ID NOs: 1-49516 and a CRISPR nuclease for inactivating a mutant RPE65 allele in a cell, comprising delivering to the cell the composition comprising an RNA molecule comprising a guide sequence portion having 17-25 contiguous nucleotides containing nucleotides in the sequence set forth in any one of SEQ ID NOs: 1-49516 and a CRISPR nuclease.

According to embodiments of the present invention, there is provided a medicament comprising an RNA molecule comprising a guide sequence portion having 17-25 contiguous nucleotides containing nucleotides in the sequence set forth in any one of SEQ ID NOs: 1-49516 and a CRISPR nuclease for use in inactivating a mutant RPE65 allele in a cell, wherein the medicament is administered by delivering to the cell the composition comprising an RNA molecule comprising a guide sequence portion having 17-25 contiguous nucleotides containing nucleotides in the sequence set forth in any one of SEQ ID NOs: 1-49516 and a CRISPR nuclease.

According to some embodiments of the present invention, there is provided use of a composition comprising an RNA molecule comprising a guide sequence portion having 17-25 contiguous nucleotides containing nucleotides in the sequence set forth in any one of SEQ ID NOs: 1-49516 and a CRISPR nuclease for treating ameliorating or preventing a dominant RPE65 gene disorder, comprising delivering to a cell of a subject having or at risk of having a dominant RPE65 gene disorder the composition of comprising an RNA molecule comprising a guide sequence portion having 17-25 contiguous nucleotides containing nucleotides in the sequence set forth in any one of SEQ ID NOs: 1-49516 and a CRISPR nuclease. In some embodiments, the method is performed ex vivo and the cell is provided/explanted from the subject. In some embodiments, the method further comprises the step of introducing the resulting cell, with the modified/knocked out mutant RPE65 allele, into the subject (e.g. autologous transplantation).

According to some embodiments of the present invention, there is provided a medicament comprising the composition comprising an RNA molecule comprising a guide sequence portion having 17-25 contiguous nucleotides containing nucleotides in the sequence set forth in any one of SEQ ID NOs: 1-49516 and a CRISPR nuclease for use in treating ameliorating or preventing a dominant RPE65 gene disorder, wherein the medicament is administered by delivering to a cell of a subject having or at risk of having a dominant RPE65 gene disorder the composition comprising an RNA molecule comprising a guide sequence portion having 17-25 contiguous nucleotides containing nucleotides in the sequence set forth in any one of SEQ ID NOs: 1-49516 and a CRISPR nuclease.

According to some embodiments of the present invention, there is provided a kit for inactivating a mutant RPE65 allele in a cell, comprising an RNA molecule comprising a guide sequence portion having 17-25 contiguous nucleotides containing nucleotides in the sequence set forth in any one of SEQ ID NOs: 1-49516, a CRISPR nuclease, and/or a tracrRNA molecule; and instructions for delivering the RNA molecule; CRISPR nuclease, and/or the tracrRNA to the cell.

According to some embodiments of the present invention, there is provided a kit for treating a dominant RPE65 gene disorder in a subject, comprising an RNA molecule comprising a guide sequence portion having 17-25 contiguous nucleotides containing nucleotides in the sequence set forth in any one of SEQ ID NOs: 1-49516, a CRISPR nuclease, and/or a tracrRNA molecule; and instructions for delivering the RNA molecule; CRISPR nuclease, and/or the tracrRNA to a cell of a subject having or at risk of having a dominant RPE65 gene disorder.

### BRIEF DESCRIPTION OF THE DRAWINGS

FIGS. 1A-B: Activity of guides targeting the p.Asp477Gly (c.1430A>G) mutation of RPE65 in patient-derived iPSCs. RNPs complexed with SpCas9 (FIG. 1A) or OMNI-50 (FIG. 1B). The nuclease and specific guide were electroporated into iPSCs to determine their activity. Cells were harvested 72 h post DNA electroporation, genomic was DNA extracted, and the region of the mutation was amplified and analyzed by capillary electrophoreses. The graphs represent the % editing ±STDV of two independent electroporation trials.

### DETAILED DESCRIPTION

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

Unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

In the description and claims of the present application, each of the verbs, “comprise,” “include” and “have” and conjugates thereof, are used to indicate that the object or objects of the verb are not necessarily a complete listing of components, elements or parts of the subject or subjects of the verb. Other terms as used herein are meant to be defined by their well-known meanings in the art.

The terms “nucleic acid template” and “donor”, refer to a nucleotide sequence that is inserted or copied into a genome. The nucleic acid template comprises a nucleotide sequence, e.g., of one or more nucleotides, that will be added to or will template a change in the target nucleic acid or may be used to modify the target sequence. A nucleic acid template sequence may be of any length, for example between 2 and 10,000 nucleotides in length, preferably between about 100 and 1,000 nucleotides in length, more preferably between about 200 and 500 nucleotides in length. A nucleic acid template may be a single stranded nucleic acid, a double stranded nucleic acid. In some embodiments, the nucleic acid template comprises a nucleotide sequence, e.g., of one or more nucleotides, that corresponds to wild type sequence of the target nucleic acid, e.g., of the target position. In some embodiments, the nucleic acid template comprises a nucleotide sequence, e.g., of one or more ribonucleotides, that corresponds to wild type sequence of the target nucleic acid, e.g., of the target position. In some embodiments, the nucleic acid template comprises modified nucleotides.

Insertion of an exogenous sequence (also called a “donor sequence,” donor template,” “donor molecule” or “donor”) can also be carried out. For example, a donor sequence can contain a non-homologous sequence flanked by two regions of homology to allow for efficient HDR at the location of interest. Additionally, donor sequences can comprise a vector molecule containing sequences that are not homologous to the region of interest in cellular chromatin. A donor molecule can contain several, discontinuous regions of homology to cellular chromatin. For example, for targeted insertion of sequences not normally present in a region of interest, said sequences can be present in a donor nucleic acid molecule and flanked by regions of homology to sequence in the region of interest. A donor molecule may be any length, for example ranging from several bases e.g. 10-20 bases to multiple kilobases in length.

The donor polynucleotide can be DNA or RNA, single-stranded and/or double-stranded and can be introduced into a cell in linear or circular form. See, e.g., U.S. Patent Publication Nos. 2010/0047805; 2011/0281361; 2011/0207221; and 2019/0330620. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al. (1987) and Nehls et al. (1996). Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

A donor sequence may be an oligonucleotide and be used for targeted alteration of an endogenous sequence. The oligonucleotide may be introduced to the cell on a vector, may be electroporated into the cell, or may be introduced via other methods known in the art. Donor polynucleotides can be introduced as naked nucleic acid, as nucleic acid complexed with an agent such as a liposome or poloxamer, or can be delivered by viruses (e.g., adenovirus, AAV, herpesvirus, retrovirus, lentivirus and integrase defective lentivirus (IDLV)).

As used herein, the term “modified cells” refers to cells in which a double strand break is affected by a complex of an RNA molecule and the CRISPR nuclease as a result of hybridization with the target sequence, i.e. on-target hybridization. The term “modified cells” may further encompass cells in which a repair or correction of a mutation was affected following the double strand break.

This invention provides a modified cell or cells obtained by use of any of the methods described herein. In an embodiment these modified cell or cells are capable of giving rise to progeny cells. In an embodiment these modified cell or cells are capable of giving rise to progeny cells after engraftment. As a non-limiting example, the modified cells may be stem cells, or any cell suitable for an allogenic cell transplant or autologous cell transplant. As a non-limiting example, the modified cell may be a stem cell. In a non-limiting example, the stem cell is an autologous pluripotent stem cell or an induced pluripotent stem cell (iPSC). As another non-limiting example, the stem cell is differentiated into a retinal pigment epithelium cell. In yet another non-limiting example, the modified cell is a retinal pigment epithelium cell.

This invention also provides a composition comprising these modified cells and a pharmaceutically acceptable carrier. Also provided is an in vitro or ex vivo method of preparing this, comprising mixing the cells with the pharmaceutically acceptable carrier.

As used herein, the term “targeting sequence” or “targeting molecule” refers a nucleotide sequence or molecule comprising a nucleotide sequence that is capable of hybridizing to a specific target sequence, e.g., the targeting sequence has a nucleotide sequence which is at least partially complementary to the sequence being targeted along the length of the targeting sequence. The targeting sequence or targeting molecule may be part of an RNA molecule that can form a complex with a CRISPR nuclease with the targeting sequence serving as the targeting portion of the CRISPR complex. When the molecule having the targeting sequence is present contemporaneously with the CRISPR molecule the RNA molecule is capable of targeting the CRISPR nuclease to the specific target sequence. Each possibility represents a separate embodiment. An RNA molecule can be custom designed to target any desired sequence.

The term “targets” as used herein, refers to a targeting sequence or targeting molecule's preferential hybridization to a nucleic acid having a targeted nucleotide sequence. It is understood that the term “targets” encompasses variable hybridization efficiencies, such that there is preferential targeting of the nucleic acid having the targeted nucleotide sequence, but unintentional off-target hybridization in addition to on-target hybridization might also occur. It is understood that where an RNA molecule targets a sequence, a complex of the RNA molecule and a CRISPR nuclease molecule targets the sequence for nuclease activity.

The “guide sequence portion” of an RNA molecule refers to a nucleotide sequence that is capable of hybridizing to a specific target DNA sequence, e.g., the guide sequence portion has a nucleotide sequence which is fully complementary to the DNA sequence being targeted along the length of the guide sequence portion. In some embodiments, the guide sequence portion is 17, 18, 19, 20, 21, 22, 23, 24, or 25 nucleotides in length, or approximately 17-25, 17-24, 17-22, 17-21, 18-25, 18-24, 18-23, 18-22, 18-21, 19-25, 19-24, 19-23, 19-22, 19-21, 19-20, 20-22, 18-20, 20-21, 21-22, or 17-20 nucleotides in length. The entire length of the guide sequence portion is fully complementary to the DNA sequence being targeted along the length of the guide sequence portion. The guide sequence portion may be part of an RNA molecule that can form a complex with a CRISPR nuclease with the guide sequence portion serving as the DNA targeting portion of the CRISPR complex. When the DNA molecule having the guide sequence portion is present contemporaneously with the CRISPR molecule the RNA molecule is capable of targeting the CRISPR nuclease to the specific target DNA sequence. Each possibility represents a separate embodiment. An RNA molecule can be custom designed to target any desired sequence.

The term “non-discriminatory” as used herein refers to a guide sequence portion of an RNA molecule that targets a specific DNA sequence that is common both a mutant and functional allele of a gene.

In embodiments of the present invention, an RNA molecule comprises a guide sequence portion having 17-25 contiguous nucleotides containing nucleotides in the sequence set forth in any one of SEQ ID NOs: 1-49516.

The RNA molecule and or the guide sequence portion of the RNA molecule may contain modified nucleotides. Exemplary modifications to nucleotides or polynucleotides may be synthetic and encompass polynucleotides which contain nucleotides comprising bases other than the naturally occurring adenine, cytosine, thymine, uracil, or guanine bases. Modifications to polynucleotides include polynucleotides which contain synthetic, non-naturally occurring nucleosides e.g., locked nucleic acids. Modifications to polynucleotides may be utilized to increase or decrease stability of an RNA. An example of a modified polynucleotide is an mRNA containing 1-methyl pseudo-uridine. For examples of modified polynucleotides and their uses, see U.S. Pat. No. 8,278,036, PCT International Publication No. WO/2015/006747, and Weissman and Kariko (2015), hereby incorporated by reference.

As used herein, “contiguous nucleotides” set forth in a SEQ ID NO refers to nucleotides in a sequence of nucleotides in the order set forth in the SEQ ID NO without any intervening nucleotides.

In embodiments of the present invention, the guide sequence portion may be at least 25 nucleotides in length and contain 20-22 contiguous nucleotides in the sequence set forth in any one of SEQ ID NOs: 1-49516. In embodiments of the present invention, the guide sequence portion may be less than 22 nucleotides in length. For example, in embodiments of the present invention the guide sequence portion may be 17, 18, 19, 20, or 21 nucleotides in length. In such embodiments the guide sequence portion may consist of 17, 18, 19, 20, or 21 nucleotides, respectively, in the sequence of 17-22 contiguous nucleotides set forth in any one of SEQ ID NOs: 1-49516. For example, a guide sequence portion having 17 nucleotides in the sequence of 17 contiguous nucleotides set forth in SEQ ID NO: 49517 may consist of any one of the following nucleotide sequences (nucleotides excluded from the contiguous sequence are marked in strike-through):

(SEQ ID NO: 49517)
AAAAAAAUGUACUUGGUUCC
17 nucleotide guide sequence 1:
(SEQ ID NO: 49518) [symbol]
AAAAUGUACUUGGUUCC
17 nucleotide guide sequence 2:
(SEQ ID NO: 49519) [symbol]
AAAAAUGUACUUGGUU [symbol]
17 nucleotide guide sequence 3:
(SEQ ID NO: 49520) [symbol]
AAAAAAUGUACUUGGUU [symbol]
17 nucleotide guide sequence 4:
(SEQ ID NO: 49521)
AAAAAAAUGUACUUGGU

[symbol]

In embodiments of the present invention, the guide sequence portion may be greater than 20 nucleotides in length. For example, in embodiments of the present invention the guide sequence portion may be 21, 22, 23, 24 or 25 nucleotides in length. In such embodiments the guide sequence portion comprises 17-25 nucleotides containing the sequence of 20, 21 or 22 contiguous nucleotides set forth in any one of SEQ ID NOs: 1-49516 and additional nucleotides fully complimentary to a nucleotide or sequence of nucleotides adjacent to the 3′ end of the target sequence, 5′ end of the target sequence, or both.

In embodiments of the present invention a CRISPR nuclease and an RNA molecule comprising a guide sequence portion form a CRISPR complex that binds to a target DNA sequence to effect cleavage of the target DNA sequence. CRISPR nucleases, e.g. Cpf1, may form a CRISPR complex comprising a CRISPR nuclease and RNA molecule without a further tracrRNA molecule. Alternatively, CRISPR nucleases, e.g. Cas9, may form a CRISPR complex between the CRISPR nuclease, an RNA molecule, and a tracrRNA molecule.

In embodiments of the present invention, the RNA molecule may further comprise the sequence of a tracrRNA molecule. Such embodiments may be designed as a synthetic fusion of the guide portion of the RNA molecule and the trans-activating crRNA (tracrRNA). (See Jinek et al., 2012). Embodiments of the present invention may also form CRISPR complexes utilizing a separate tracrRNA molecule and a separate RNA molecule comprising a guide sequence portion. In such embodiments the tracrRNA molecule may hybridize with the RNA molecule via basepairing and may be advantageous in certain applications of the invention described herein.

The term “tracr mate sequence” refers to a sequence sufficiently complementary to a tracrRNA molecule so as to hybridize to the tracrRNA via basepairing and promote the formation of a CRISPR complex. (See U.S. Pat. No. 8,906,616). In embodiments of the present invention, the RNA molecule may further comprise a portion having a tracr mate sequence.

A “gene,” for the purposes of the present disclosure, includes a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Eukaryotic” cells include, but are not limited to, fungal cells (such as yeast), plant cells, animal cells, mammalian cells and human cells.

The term “nuclease” as used herein refers to an enzyme capable of cleaving the phosphodiester bonds between the nucleotide subunits of nucleic acid. A nuclease may be isolated or derived from a natural source. The natural source may be any living organism. Alternatively, a nuclease may be a modified or a synthetic protein which retains the phosphodiester bond cleaving activity. Gene modification can be achieved using a nuclease, for example a CRISPR nuclease.

As used herein, “progenitor cell” refers to a lineage cell that is derived from stem cell and retains mitotic capacity and multipotency (e.g., can differentiate or develop into more than one but not all types of mature lineage of cell).

The term “single nucleotide polymorphism (SNP) position”, as used herein, refers to a position in which a single nucleotide DNA sequence variation occurs between members of a species, or between paired chromosomes in an individual. In the case that a SNP position exists at paired chromosomes in an individual, a SNP on one of the chromosomes is a “heterozygous SNP.” The term SNP position refers to the particular nucleic acid position where a specific variation occurs and encompasses both a sequence including the variation from the most frequently occurring base at the particular nucleic acid position (also referred to as “SNP” or alternative “ALT”) and a sequence including the most frequently occurring base at the particular nucleic acid position (also referred to as reference, or “REF”). Accordingly, the sequence of a SNP position may reflect a SNP (i.e. an alternative sequence variant relative to a consensus reference sequence within a population), or the reference sequence itself.

According to embodiments of the present invention, there is provided a method for modifying in a cell a mutant allele of the retinal pigment epithelium-specific 65 kDa protein gene (RPE65) gene having a mutation associated with a dominant RPE65 gene disorder, the method comprising
introducing to the cell a composition comprising:
   at least one CRISPR nuclease or a sequence encoding a CRISPR nuclease; and
   a first RNA molecule comprising a guide sequence portion having 17-25 nucleotides or a nucleotide sequence encoding the same,
wherein a complex of the CRISPR nuclease and the first RNA molecule affects a double strand break in the mutant allele of the RPE65 gene.

In some embodiments, the first RNA molecule targets the CRISPR nuclease to the mutation associated with a dominant RPE65 gene disorder.

In some embodiments, the mutation associated with a dominant RPE65 gene disorder is any one of 1:68431085_T_C and 1:68446825_G_A.

In some embodiments, the guide sequence portion of the first RNA molecule comprises 17-25 contiguous nucleotides containing nucleotides in the sequence set forth in any one of SEQ ID NOs: 1-49516 that targets a mutation associated with a dominant RPE65 gene disorder.

In some embodiments, the first RNA molecule targets the CRISPR nuclease to a SNP position of the mutant allele.

In some embodiments, the SNP position is any one of rs60701104, rs9436400, rs868541802, rs3125890, rs75159457, rs1205919238, rs11209300, rs4264030, rs2419988, rs3118415, rs3118416, rs149739986, rs2182315, rs3118418, rs932783, rs12124063, rs77585943, rs1886906, rs3125891, rs11581095, rs12030710, rs1003041423, rs3118419, rs5774935, rs150459448, rs1555845, rs1555846, rs11269074, rs3790469, rs3125894, rs3125895, rs3125896, rs3125897, rs3125898, rs17130688, rs938759267, rs34194247, rs3125900, rs79716012, rs75711879, rs12145904, rs3125902, rs3118420, rs12138573, rs1925955, rs17130691, rs3125904, rs78507000, rs150774295, rs3125905, rs2038900, rs2038901, rs147665807, rs17130694, rs12408546, rs12077372, rs3790472, rs3790473, rs147893529, rs2012235, rs3118423, rs2986125, rs2986124, rs72926973, rs2277874, rs3125906, rs3118426, rs3125907, rs382422, rs3118427, rs3125908, rs12759602, rs2477974, rs3125909, rs3118428, rs12407140, rs72674322, rs72674323, rs3125910, and rs1318744874.

In some embodiments, the guide sequence portion of the first RNA molecule comprises 17-25 contiguous nucleotides containing nucleotides in the sequence set forth in any one of SEQ ID NOs: 1-49516 that targets a SNP position of the mutant allele.

In some embodiments, the SNP position is in an exon or intron of the RPE65 mutant allele.

In some embodiments, the SNP position contains a heterozygous SNP.

In some embodiments, the method further comprises introducing to the cell a second RNA molecule comprising a guide sequence portion having 17-25 nucleotides or a nucleotide sequence encoding the same, wherein a complex of the second RNA molecule and a CRISPR nuclease affects a second double strand break in the RPE65 gene.

In some embodiments, the guide sequence portion of the second RNA molecule comprises 17-25 contiguous nucleotides containing nucleotides in the sequence set forth in any one of SEQ ID NOs: 1-49516 other than the sequence of the first RNA molecule.

In some embodiments, the second RNA molecule comprises a non-discriminatory guide portion that targets both functional and mutated RPE65 alleles.

In some embodiments, the second RNA molecule comprises a non-discriminatory guide portion that targets any one of Intron 1 of RPE65, Intron 2 of RPE65, a 3′ untranslated region (3′ UTR) of RPE65, and an intergenic region downstream of RPE65.

In some embodiments, the second RNA molecule comprises a non-discriminatory guide portion that targets a sequence that is located within a genomic range selected from any one of 1:68450655-1:68451154, 1:68428322-1:68428821, 1:68437687-1:68438186, 1:68431586-1:68432085, 1:68431377-1:68431469, 1:68431177-1:68431281, 1:68430565-1:68431064, 1:68429928-1:68430427, 1:68448707-1:68449206, 1:68449395-1:68449894, 1:68448124-1:68448623, 1:68446861-1:68447360, 1:68446210-1:68446709, 1:68444884-1:68445383, 1:68444673-1:68444775, 1:68444031-1:68444530, 1:68441001-1:68441500, 1:68440353-1:68440852, 1:68439643-1:68440142, 1:68439324-1:68439560, 1:68439082-1:68439190, and 1:68438317-1:68438941.

In some embodiments, the second RNA molecule comprises a non-discriminatory guide portion that targets a sequence that is located up to 500 base pairs from an exon that is excised by the first and second RNA molecules.

In some embodiments, a portion of an exon is excised from the mutant allele of the RPE65 gene.

In some embodiments, the first RNA molecule targets a SNP position in the 3′ UTR of the mutated allele, and the second RNA molecule comprises a non-discriminatory guide portion that targets downstream of a polyadenylation signal sequence that is common to both a functional allele and the mutant allele of the RPE65 gene.

In some embodiments, the first RNA molecule targets a SNP position downstream of a polyadenylation signal of the mutated allele, and the second RNA molecule comprises a non-discriminatory guide portion that targets a sequence upstream of a polyadenylation signal that is common to both a functional allele and the mutant allele of the RPE65 gene.

In some embodiments, the polyadenylation signal is excised from the mutant allele of the RPE65 gene.

According to embodiments of the present invention, there is provided a modified cell obtained by the method of any one of the embodiments presented herein.

According to embodiments of the present invention, there is provided a first RNA molecule comprising a guide sequence portion having 17-25 contiguous nucleotides containing nucleotides in the sequence set forth in any one of SEQ ID NOs: 1-49516.

According to embodiments of the present invention, there is provided a composition comprising the first RNA molecule and at least one CRISPR nuclease.

In some embodiments, the composition further comprises a second RNA molecule comprising a guide sequence portion having 17-25 contiguous nucleotides, wherein the second RNA molecule targets a RPE65 allele, and wherein the guide sequence portion of the second RNA molecule is a different sequence from the sequence of the guide sequence portion of the first RNA molecule.

In some embodiments, the guide sequence portion of the second RNA molecule comprises 17-25 contiguous nucleotides containing nucleotides in the sequence set forth in any one of SEQ ID NOs: 1-49516 other than the sequence of the first RNA molecule.

According to embodiments of the present invention, there is provided a method for inactivating a mutant RPE65 allele in a cell, the method comprising delivering to the cell the composition of any one of the embodiments presented herein.

According to embodiments of the present invention, there is provided a method for treating a dominant RPE65 gene disorder, the method comprising delivering to a cell of a subject having a dominant RPE65 gene disorder the composition of any one of the embodiments presented herein.

According to embodiments of the present invention, there is provided use of any one of the compositions presented herein for inactivating a mutant RPE65 allele in a cell, comprising delivering to the cell the composition of any one of the embodiments presented herein.

According to embodiments of the present invention, there is provided a medicament comprising the composition of any one of the embodiments presented herein for use in inactivating a mutant RPE65 allele in a cell, wherein the medicament is administered by delivering to the cell the composition of any one of the embodiments presented herein.

According to embodiments of the present invention, there is provided use of the composition of any one of the embodiments presented herein for treating ameliorating or preventing a dominant RPE65 gene disorder, comprising delivering to a cell of a subject having or at risk of having a dominant RPE65 gene disorder the composition of any one of the embodiments presented herein.

According to embodiments of the present invention, there is provided a medicament comprising the composition of any one of the embodiments presented herein for use in treating ameliorating or preventing a dominant RPE65 gene disorder, wherein the medicament is administered by delivering to a cell of a subject having or at risk of having a dominant RPE65 gene disorder the composition of any one of the embodiments presented herein.

According to embodiments of the present invention, there is provided a kit for inactivating a mutant RPE65 allele in a cell, comprising an RNA molecule of any one of the embodiments presented herein, a CRISPR nuclease, and/or a tracrRNA molecule; and instructions for delivering the RNA molecule; CRISPR nuclease, and/or the tracrRNA to the cell.

According to embodiments of the present invention, there is provided a kit for treating a dominant RPE65 gene disorder in a subject, comprising an RNA molecule of any one of the embodiments presented herein, a CRISPR nuclease, and/or a tracrRNA molecule; and instructions for delivering the RNA molecule; CRISPR nuclease, and/or the tracrRNA to a cell of a subject having or at risk of having a dominant RPE65 gene disorder.

According to embodiments of the present invention, there is provided a gene editing composition comprising an RNA molecule comprising a guide sequence portion having 17-25 contiguous nucleotides containing nucleotides in the sequence set forth in any one of SEQ ID NOs: 1-49516. In some embodiments, the RNA molecule further comprises a portion having a sequence which binds to a CRISPR nuclease. In some embodiments, the sequence which binds to a CRISPR nuclease is a tracrRNA sequence.

In some embodiments, the RNA molecule further comprises a portion having a tracr mate sequence.

In some embodiments, the RNA molecule may further comprise one or more linker portions.

According to embodiments of the present invention, an RNA molecule may be up to 300, 290, 280, 270, 260, 250, 240, 230, 220, 210, 200, 190, 180, 170, 160, 150, 140, 130, 120, 110, or 100 nucleotides in length. Each possibility represents a separate embodiment. In embodiments of the present invention, the RNA molecule may be 17 up to 300 nucleotides in length, 100 up to 300 nucleotides in length, 150 up to 300 nucleotides in length, 200 up to 300 nucleotides in length, 100 to 200 nucleotides in length, or 150 up to 250 nucleotides in length. Each possibility represents a separate embodiment.

According to some embodiments of the present invention, the composition further comprises a tracrRNA molecule.

The present disclosure provides a method for utilizing at least one naturally occurring nucleotide difference or polymorphism (e.g., single nucleotide polymorphism (SNP)) for distinguishing/discriminating between two alleles of a gene, one allele bearing a mutation such that it encodes a mutated protein causing a disease phenotype (“mutated allele”) and a particular sequence in a SNP position (SNP/REF), and the other allele encoding for a functional protein (“functional allele”). The method further comprises the step of knocking out expression of the mutated protein and allowing expression of the functional protein. In some embodiments, the method is for treating, ameliorating, or preventing a dominant negative genetic disorder.

According to some embodiments of the present invention, there is provided a method for inactivating a mutant RPE65 allele in a cell, the method comprising delivering to the cell a composition comprising an RNA molecule comprising a guide sequence portion having 17-25 contiguous nucleotides containing nucleotides in the sequence set forth in any one of SEQ ID NOs: 1-49516 and a CRISPR nuclease.

According to some embodiments of the present invention, there is provided a method for treating a dominant RPE65 gene disorder, the method comprising delivering to a cell of a subject having a dominant RPE65 gene disorder a composition comprising an RNA molecule comprising a guide sequence portion having 17-25 contiguous nucleotides containing nucleotides in the sequence set forth in any one of SEQ ID NOs: 1-49516 and a CRISPR nuclease.

According to embodiments of the present invention, the composition comprises a second RNA molecule comprising a guide sequence portion having 17-25 contiguous nucleotides containing nucleotides in the sequence set forth in any one of SEQ ID NOs: 1-49516. In some embodiments, the 17-25 nucleotides of the guide sequence portion of the second RNA molecule are in a different sequence from the sequence of the guide sequence portion of the first RNA molecule.

According to embodiments of the present invention, at least one CRISPR nuclease and the RNA molecule or RNA molecules are delivered to the subject and/or cells substantially at the same time or at different times.

In some embodiments, a tracrRNA molecule is delivered to the subject and/or cells substantially at the same time or at different times as the CRISPR nuclease and RNA molecule or RNA molecules.

According to embodiments of the present invention, the first RNA molecule targets a SNP or disease-causing mutation in the exon or promoter of a mutated allele, and the second RNA molecule targets a SNP in an exon of the mutated allele, a SNP in an intron, or a sequence present in both the mutated or functional allele.

According to embodiments of the present invention, the first RNA molecule or the first and the second RNA molecules target a SNP in the promoter region, the start codon, or an untranslated region (UTR) of a mutated allele.

According to embodiments of the present invention, the first RNA molecule or the first and the second RNA molecules targets at least a portion of the promoter and/or the start codon and/or a portion of a UTR of a mutated allele.

According to embodiments of the present invention, the first RNA molecule targets a portion of the promoter, a first SNP in the promoter, or a SNP upstream to the promoter of a mutated allele and the second RNA molecule is targets a second SNP, which is downstream of the first SNP, and is in the promoter, in a UTR, or in an intron or in an exon of a mutated allele.

According to embodiments of the present invention, the first RNA molecule targets a SNP in the promoter, upstream of the promoter, or a UTR of a mutated allele and the second RNA molecule is designed to target a sequence which is present in an intron of both the mutated allele and the functional allele.

According to embodiments of the present invention, the first RNA molecule targets a SNP in an intron of a mutated allele, and wherein the second RNA molecule targets a SNP in an intron of the mutated allele, or a sequence in an intron present in both the mutated and functional allele.

According to embodiments of the present invention, the first RNA molecule targets a sequence upstream of the promotor which is present in both a mutated and functional allele and the second RNA molecule targets a SNP or disease-causing mutation in any location of the gene.

According to embodiments of the present invention, there is provided a method comprising removing an exon containing a disease-causing mutation from a mutated allele, wherein the first RNA molecule or the first and the second RNA molecules target regions flanking an entire exon or a portion of the exon.

According to embodiments of the present invention, there is provided a method comprising removing an exon or a portion thereof from a mutant RPE65 allele, the entire open reading frame of a mutant RPE65 allele, or removing the entire mutant RPE65 allele.

According to embodiments of the present invention, the first RNA molecule targets a SNP or disease-causing mutation in an exon or promoter of a mutated allele, and wherein the second RNA molecule targets a SNP in the same exon of the mutated allele, a SNP in an intron, or a sequence in an intron present in both the mutated and functional allele.

According to embodiments of the present invention, the first RNA molecule or the first and the second RNA molecules target an alternative splicing signal sequence between an exon and an intron of a mutant RPE65 allele.

According to embodiments of the present invention, the second RNA molecule is non-discriminatory targets a sequence present in both a mutated allele and a functional allele.

The compositions and methods of the present disclosure may be utilized for treating, preventing, ameliorating, or slowing progression of an autosomal dominant genetic disorder, such as a dominant RPE65 gene disorder.

In some embodiments, a mutated allele is deactivated by delivering to a cell an RNA molecule which targets a SNP in the promoter region, the start codon, or an untranslated region (UTR) of the mutated allele.

In some embodiments, a mutated allele is inactivated by removing at least a portion of the promoter, and/or removing the start codon, and/or a portion of the UTR, and/or a polyadenylation signal. In such embodiments one RNA molecule may be designed for targeting a first SNP in the promoter or upstream to the promoter and another RNA molecule is designed to target a second SNP, which is downstream of the first SNP, and is in the promoter, in the UTR, in an intron, or in an exon. Alternatively, one RNA molecule may be designed for targeting a SNP in the promoter, upstream of the promoter, or the UTR, and another RNA molecule is designed to target a sequence which is present in an intron of both the mutated allele and the functional allele.

Alternatively, one RNA molecule may be designed for targeting a sequence upstream of the promotor which is present in both the mutated and functional allele and the other guide is designed to target a SNP or disease-causing mutation in any location of the gene e.g., in an exon, intron, UTR, or downstream of the promoter.

In some embodiments, the method of deactivating a mutated allele comprises an exon skipping step comprising removing an exon containing a disease-causing mutation from the mutated allele. Removing an exon containing a disease-causing mutation in the mutated allele requires two RNA molecules which target regions flanking the entire exon or a portion of the exon. Removal of an exon containing the disease-causing mutation may be designed to eliminate the disease-causing action of the protein while allowing for expression of the remaining protein product which retains some or all of the wild-type activity. The entire open reading frame or the entire gene can be excised using two RNA molecules flanking the region desired to be excised.

In some embodiments, the method of deactivating a mutated allele comprises delivering two RNA molecules to a cell, wherein one RNA molecule targets a SNP or disease-causing mutation in an exon or promoter of the mutated allele, and wherein the other RNA molecule targets a SNP in the same of the mutated allele, a SNP in an intron, or a sequence in an intron present in both the mutated or functional allele.

Any one of, or combination of, the above-mentioned strategies for deactivating a mutant allele may be used in the context of the invention.

In embodiments of the present invention, an RNA molecule is used to direct a CRISPR nuclease to an exon or a splice site of a mutated allele in order to create a double-stranded break (DSB), leading to insertion or deletion of nucleotides by inducing an error-prone non-homologous end-joining (NHEJ) mechanism and formation of a frameshift mutation in the mutated allele. The frameshift mutation may result in, for example, inactivation or knockout of the mutated allele by generation of an early stop codon in the mutated allele and to generation of a truncated protein or to nonsense-mediated mRNA decay of the transcript of the mutant allele. In further embodiments, one RNA molecule is used to direct a CRISPR nuclease to a promotor of a mutated allele.

In some embodiments, the method of deactivating a mutated allele further comprises enhancing activity of the functional protein such as by providing a protein/peptide, a nucleic acid encoding a protein/peptide, or a small molecule such as a chemical compound, capable of activating/enhancing activity of the functional protein.

According to some embodiments, the present disclosure provides an RNA sequence (also referred to as an ‘RNA molecule’) which binds to or associates with and/or directs an RNA-guided DNA nuclease e.g., a CRISPR nuclease, to a target sequence comprising at least one nucleotide which differs between a mutated allele and a functional allele (e.g., SNP) of a gene of interest (i.e., a sequence of the mutated allele which is not present in the functional allele).

In some embodiments, the method comprises contacting a mutated allele of a gene of interest with an allele-specific RNA molecule and a CRISPR nuclease e.g., a Cas9 protein, wherein the allele-specific RNA molecule and the CRISPR nuclease associate with a nucleotide sequence of the mutated allele of the gene of interest which differs by at least one nucleotide from a nucleotide sequence of a functional allele of the gene of interest, thereby modifying or knocking-out the mutated allele.

In some embodiments, the allele-specific RNA molecule and a CRISPR nuclease is introduced to a cell encoding the gene of interest. In some embodiments, the cell encoding the gene of interest is in a mammalian subject. In some embodiments, the cell encoding the gene of interest is in a plant.

In some embodiments, the mutated allele is an allele of RPE65 gene. In some embodiments, the RNA molecule targets a SNP which co-exists with or is genetically linked to the mutated sequence associated with a dominant RPE65 gene disorder genetic disorder. In some embodiments, the RNA molecule targets a SNP which is highly prevalent in the population and exists in the mutated allele having the mutated sequence associated with a dominant RPE65 gene disorder genetic disorder and not in the functional allele of an individual subject to be treated. In some embodiments, a disease-causing mutation within a mutated RPE65 allele is targeted.

In some embodiments, the SNP is within an exon of the gene of interest. In such embodiments, a guide sequence portion of an RNA molecule is designed to associate with a sequence of an exon of the gene of interest.

In some embodiments, SNP is within an intron or the exon of the gene of interest. In some embodiments, the SNP is in close proximity to the splice site between an intron and an exon. In some embodiments, the close proximity to a splice site is 1, 2, 3, 4, 5, 6, 7, 8, 9, 10, 11, 12, 13, 14, 15, 16, 17, 18, 19, or 20 nucleotides upstream or downstream to the splice site. Each possibility represents a separate embodiment of the present invention. In such embodiments, a guide sequence portion of an RNA molecule may be designed to associate with a sequence of the gene of interest which comprises the splice site.

In some embodiments, the method is utilized for treating a subject having a disease phenotype resulting from the heterozygote RPE65 gene. In such embodiments, the method results in improvement, amelioration or prevention of the disease phenotype.

Embodiments of compositions described herein include at least one CRISPR nuclease, RNA molecule(s), and a tracrRNA molecule, being effective in a subject or cells at the same time. The at least one CRISPR nuclease, RNA molecule(s), and tracrRNA may be delivered substantially at the same time or can be delivered at different times but have effect at the same time. For example, this includes delivering the CRISPR nuclease to the subject or cells before the RNA molecule and/or tracrRNA is substantially extant in the subject or cells.

In some embodiments, the cell is a stem cell. In some embodiments, the stem cell is an autologous pluripotent stem cell or an induced pluripotent stem cell (iPSC). In some embodiments, the stem cell is differentiated into a retinal pigment epithelium cell. In some embodiments, the cell is a retinal pigment epithelium cell.

### Dominant Genetic Disorders

One of skill in the art will appreciate that all subjects with any type of heterozygote genetic disorder (e.g., dominant genetic disorder) may be subjected to the methods described herein. In one embodiment, the present invention may be used to target a gene involved in, associated with, or causative of dominant genetic disorders such as, for example, a dominant RPE65 gene disorder. In some embodiments, the dominant genetic disorder is a dominant RPE65 gene disorder. In some embodiments, the target gene is the RPE65 gene. Non-limiting examples of mutations characterized as gain of function mutations associated with a dominant RPE65 gene disorder phenotype include chr:1:68431085(hg398) T to C (c.1430A>G; p.D477G) and chr1:68446825(hg38) G to A (c.130C>T; p.R44X).

RPE65 editing strategies include, but are not limited to, (1) truncation, for example, by targeting a RPE65 mutation or SNP position with one guide RNA molecule to induce a frameshift or nonsense-mediated decay; and (2) allele specific excision using two guide RNA molecules, for example, excision of at least one exon or a portion thereof, knockout of a large portion of the allele or the entire allele, or excision of the polyadenylation signal.

Truncation may be achieved by several approaches. For example, truncation may be achieved by targeting a SNP within a coding exon of a mutant RPE65 allele using a single guide RNA molecule (e.g. a single guide RNA molecule or “sgRNA”). Alternatively, excision may be achieved by targeting the mutant RPE65 allele with two different RNA molecules, with at least one RNA molecule preferably being allele-specific.

In another editing strategy, expression of a mutated RPE65 allele may be inhibited. An example of this strategy includes excising the polyadenylation signal in the 3′UTR region, which leads to an unstable transcript.

### CRISPR Nucleases and PAM Recognition

In some embodiments, the sequence specific nuclease is selected from CRISPR nucleases, or a functional variant thereof. In some embodiments, the sequence specific nuclease is an RNA guided DNA nuclease. In such embodiments, the RNA sequence which guides the RNA guided DNA nuclease (e.g., Cpf1) binds to and/or directs the RNA guided DNA nuclease to the sequence comprising at least one nucleotide which differs between a mutated allele and its counterpart functional allele (e.g., SNP). In some embodiments, the CRISPR complex does not further comprise a tracrRNA. In a non-limiting example, in which the RNA guided DNA nuclease is a CRISPR protein, the at least one nucleotide which differs between the dominant mutated allele and the functional allele may be within the PAM site and/or proximal to the PAM site within the region that the RNA molecule is designed to hybridize to. A skilled artisan will appreciate that RNA molecules can be engineered to bind to a target of choice in a genome by commonly known methods in the art.

In embodiments of the present invention, a type II CRISPR system utilizes a mature crRNA:tracrRNA complex directs a CRISPR nuclease, e.g. Cas9, to the target DNA via Watson-Crick base-pairing between the crRNA and the protospacer on the target DNA next to the protospacer adjacent motif (PAM), an additional requirement for target recognition. The CRISPR nuclease then mediates cleavage of target DNA to create a double-stranded break within the protospacer. A skilled artisan will appreciate that each of the engineered RNA molecule of the present invention is further designed such as to associate with a target genomic DNA sequence of interest next to a protospacer adjacent motif (PAM), e.g., a PAM matching the sequence relevant for the type of CRISPR nuclease utilized, such as for a non-limiting example, NGG or NAG, wherein “N” is any nucleobase, for Streptococcus pyogenes Cas9 WT (SpCAS9); NNGRRT for Staphylococcus aureus (SaCas9); NNNVRYM for Jejuni Cas9 WT; NGAN or NGNG for SpCas9-VQR variant; NGCG for SpCas9-VRER variant; NGAG for SpCas9-EQR variant; NNNNGATT for Neisseria meningitidis (NmCas9); or TTV for Cpf1. RNA molecules of the present invention are each designed to form complexes in conjunction with one or more different CRISPR nucleases and designed to target polynucleotide sequences of interest utilizing one or more different PAM sequences respective to the CRISPR nuclease utilized.

In some embodiments, an RNA-guided DNA nuclease e.g., a CRISPR nuclease, may be used to cause a DNA break, either double or single-stranded in nature, at a desired location in the genome of a cell. The most commonly used RNA-guided DNA nucleases are derived from CRISPR systems, however, other RNA-guided DNA nucleases are also contemplated for use in the genome editing compositions and methods described herein. For instance, see U.S. Patent Publication No. 2015/0211023, incorporated herein by reference.

CRISPR systems that may be used in the practice of the invention vary greatly. CRISPR systems can be a type I, a type II, or a type III system. Non-limiting examples of suitable CRISPR proteins include Cas3, Cas4, Cas5, Cas5e (or CasD), Cas6, Cas6e, Cas6f, Cas7, Cas8a1, Cas8a2, Cas8b, Cas8c, Cas9, Casl0, Casl Od, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3,Csxl7, Csxl4, Csxl0, Csxl6, CsaX, Csx3, Cszl, Csxl5, Csfl, Csf2, Csf3, Csf4, and Cul966.

In some embodiments, the RNA-guided DNA nuclease is a CRISPR nuclease derived from a type II CRISPR system (e.g., Cas9). The CRISPR nuclease may be derived from Streptococcus pyogenes, Streptococcus thermophilus, Streptococcus sp., Staphylococcus aureus. Neisseria meningitidis, Treponema denticola, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonmfex degensii, Caldicelulosiruptor becscii, Candidatus Desulforudis, Clostridium botulinum, Clostridium difjicile, Finegoldia magna, Natranaerobius thermophilus, Pelotomaculumthermopropionicum. Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, or any species which encodes a CRISPR nuclease with a known PAM sequence. CRISPR nucleases encoded by uncultured bacteria may also be used in the context of the invention. (See Burstein et al. Nature, 2017). Variants of CRIPSR proteins having known PAM sequences e.g., SpCas9 D1135E variant, SpCas9 VQR variant, SpCas9 EQR variant, or SpCas9 VRER variant may also be used in the context of the invention.

Thus, an RNA guided DNA nuclease of a CRISPR system, such as a Cas9 protein or modified Cas9 or homolog or ortholog of Cas9, or other RNA guided DNA nucleases belonging to other types of CRISPR systems, such as Cpf1 and its homologs and orthologs, may be used in the compositions of the present invention.

In certain embodiments, the CRIPSR nuclease may be a “functional derivative” of a naturally occurring Cas protein. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof. Suitable derivatives of a Cas polypeptide or a fragment thereof include but are not limited to mutants, fusions, covalent modifications of Cas protein or a fragment thereof. Cas protein, which includes Cas protein or a fragment thereof, as well as derivatives of Cas protein or a fragment thereof, may be obtainable from a cell or synthesized chemically or by a combination of these two procedures. The cell may be a cell that naturally produces Cas protein, or a cell that naturally produces Cas protein and is genetically engineered to produce the endogenous Cas protein at a higher expression level or to produce a Cas protein from an exogenously introduced nucleic acid, which nucleic acid encodes a Cas that is same or different from the endogenous Cas. In some cases, the cell does not naturally produce Cas protein and is genetically engineered to produce a Cas protein.

In some embodiments, the CRISPR nuclease is Cpf1. Cpf1 is a single RNA-guided endonuclease which utilizes a T-rich protospacer-adjacent motif. Cpf1 cleaves DNA via a staggered DNA double-stranded break. Two Cpf1 enzymes from Acidaminococcus and Lachnospiraceae have been shown to carry out efficient genome-editing activity in human cells. (See Zetsche et al., 2015).

Thus, an RNA guided DNA nuclease of a Type II CRISPR System, such as a Cas9 protein or modified Cas9 or homologs, orthologues, or variants of Cas9, or other RNA guided DNA nucleases belonging to other types of CRISPR systems, such as Cpf1 and its homologs, orthologues, or variants, may be used in the present invention.

In some embodiments, the guide molecule comprises one or more chemical modifications which imparts a new or improved property (e.g., improved stability from degradation, improved hybridization energetics, or improved binding properties with an RNA guided DNA nuclease). Suitable chemical modifications include, but are not limited to: modified bases, modified sugar moieties, or modified inter-nucleoside linkages. Non-limiting examples of suitable chemical modifications include: 4-acetylcytidine, 5-(carboxyhydroxymethyl)uridine, 2′-O-methylcytidine, 5-carboxymethylaminomethyl-2-thiouridine, 5-carboxymethylaminomethyluridine, dihydrouridine, 2′-O-methylpseudouridine, “beta, D-galactosylqueuosine”, 2′-O-methylguanosine, inosine, N6-isopentenyladenosine, 1-methyladenosine, 1-methylpseudouridine, 1-methylguanosine, 1-methylinosine, “2,2-dimethylguanosine”, 2-methyladenosine, 2-methylguanosine, 3-methylcytidine, 5-methylcytidine, N6-methyladenosine, 7-methylguanosine, 5-methylaminomethyluridine, 5-methoxyaminomethyl-2-thiouridine, “beta, D-mannosylqueuosine”, 5-methoxycarbonylmethyl-2-thiouridine, 5-methoxycarbonylmethyluridine, 5-methoxyuridine, 2-methylthio-N6-isopentenyladenosine, N-((9-beta-D-ribofuranosyl-2-methylthiopurine-6-yl)carbamoyl)threonine, N-((9-beta-D-ribofuranosylpurine-6-yl)N-methylcarbamoyl)threonine, uridine-5-oxyacetic acid-methylester, uridine-5-oxyacetic acid, wybutoxosine, queuosine, 2-thiocytidine, 5-methyl-2-thiouridine, 2-thiouridine, 4-thiouridine, 5-methyluridine, N-((9-beta-D-ribofuranosylpurine-6-yl)-carbamoyl)threonine, 2′-O-methyl-5-methyluridine, 2′-O-methyluridine, wybutosine, “3-(3-amino-3-carboxy-propyl)uridine, (acp3)u”, 2′-0-methyl (M), 3′-phosphorothioate (MS), 3-thioPACE (MSP), pseudouridine, or 1-methyl pseudo-uridine. Each possibility represents a separate embodiment of the present invention.

Guide Sequences which Specifically Target a Mutant Allele

A given gene may contain thousands of SNPs. Utilizing a twenty-five base pair target window for targeting each SNP in a gene would require hundreds of thousands of guide sequences. Any given guide sequence when utilized to target a SNP may result in degradation of the guide sequence, limited activity, no activity, or off-target effects. Accordingly, suitable guide sequences are necessary for targeting a given gene. By the present invention, a novel set of guide sequences have been identified for knocking out expression of a mutated RPE65 protein, inactivating a mutant RPE65 gene allele, and treating a dominant RPE65 gene disorder.

The present disclosure provides guide sequences capable of specifically targeting a mutated allele for inactivation while leaving the functional allele unmodified. The guide sequences of the present invention are designed to, and are most likely to, specifically differentiate between a mutated allele and a functional allele. Of all possible guide sequences which target a mutated allele desired to be inactivated, the specific guide sequences disclosed herein are specifically effective to function with the disclosed embodiments.

Briefly, the guide sequences may have properties as follows: (1) target SNP/insertion/deletion/indel with a high prevalence in the general population, in a specific ethnic population or in a patient population is above 1% and the SNP/insertion/deletion/indel heterozygosity rate in the same population is above 1%; (2) target a location of a SNP/insertion/deletion/indel proximal to a portion of the gene e.g., within 5 k bases of any portion of the gene, for example, a promoter, a UTR, an exon or an intron; and (3) target a mutant allele using an RNA molecule which targets a founder or common pathogenic mutations for the disease/gene. In some embodiments, the prevalence of the SNP/insertion/deletion/indel in the general population, in a specific ethnic population or in a patient population is above 1%, 2%, 3%, 4%, 5%, 6%, 7%, 8%, 9%, 10%, 11%, 12%, 13%, 14%, or 15% and the SNP/insertion/deletion/indel heterozygosity rate in the same population is above 1%, 2%, 3%, 4%, 5%, 6%, 7%, 8%, 9%, 10%, 11%, 12%, 13%, 14%, or 15%. Each possibility represents a separate embodiment and may be combined at will.

For each gene, according to SNP/insertion/deletion/indel any one of the following strategies may be used to deactivate the mutated allele: (1) Knockout strategy using one RNA molecule—one RNA molecule is utilized to direct a CRISPR nuclease to a mutated allele and create a double-strand break (DSB) leading to formation of a frameshift mutation in an exon or in a splice site region of the mutated allele; and (2) Excision of at least one coding exon or a complete knockout of a mutant RPE65 allele using two RNA molecules, for example, a first RNA molecule targets a SNP position of an Intron 1 of the mutant RPE65 allele and a second, non-discriminatory RNA molecule targets a sequence in Intron 2 of the RPE65 gene.

Based on the locations of identified SNPs/insertions/deletions/indels for each mutant allele, any one of, or a combination of, the above-mentioned methods to deactivate the mutant allele may be utilized.

In some embodiments of the present invention, an RNA molecule is used to target a pathogenic mutation within a mutant RPE65 allele. In some embodiments of the present invention, an RNA molecule is used to target a SNP position.

Guide sequences of the present invention may: (1) target a heterozygous SNP for the targeted gene; (2) target a heterozygous SNP upstream or downstream of the gene; (3) target a SNP with a prevalence of the SNP/insertion/deletion/indel in the general population, in a specific ethnic population, or in a patient population above 1%; (4) have a guanine-cytosine content of greater than 30% and less than 85%; (5) have no repeat of seven or more guanine, cytosine, uracil, or adenine; and (6) have low or no off-targeting identified by off-target analysis. Guide sequences of the present invention may satisfy any one of the above criteria and are most likely to differentiate between a mutated allele from its corresponding functional allele.

In some embodiments of the present invention, at least one nucleotide which differs between the mutated allele and the functional allele is upstream, downstream or within the sequence of the disease-causing mutation of the gene of interest. The at least one nucleotide which differs between the mutated allele and the functional allele may be within an exon or within an intron of the gene of interest. In some embodiments, the at least one nucleotide which differs between the mutated allele and the functional allele is within an exon of the gene of interest. In some embodiments, the at least one nucleotide which differs between the mutated allele and the functional allele is within an intron or the exon of the gene of interest, in close proximity to the splice site between the intron and the exon e.g., 1, 2, 3, 4, 5, 6, 7, 8, 9, 10, 11, 12, 13, 14, 15, 16, 17, 18, 19, or 20 nucleotides upstream or downstream to the splice site.

In some embodiments, the at least one nucleotide is a single nucleotide polymorphism (SNP). In some embodiments, each of the nucleotide variants of the SNP may be expressed in the mutated allele. In some embodiments, the SNP may be a founder or common pathogenic mutation.

Guide sequences may target a SNP which has both (1) a high prevalence in the general population e.g., above 1% in the population; and (2) a high heterozygosity rate in the population, e.g., above 1%. Guide sequences may target a SNP that is globally distributed. A SNP may be a founder or common pathogenic mutation. In some embodiments, the prevalence in the general population is above 1%, 2%, 3%, 4%, 5%, 6%, 7%, 8%, 9%, 10%, 11%, 12%, 13%, 14%, or 15%. Each possibility represents a separate embodiment. In some embodiments, the heterozygosity rate in the population is above 1%, 2%, 3%, 4%, 5%, 6%, 7%, 8%, 9%, 10%, 11%, 12%, 13%, 14%, or 15%. Each possibility represents a separate embodiment.

In some embodiments, the at least one nucleotide which differs between the mutated allele and the functional allele is linked to/co-exists with the disease-causing mutation in high prevalence in a population. In such embodiments, “high prevalence” refers to at least 92%, 93%, 94%, 95%, 96%, 97%, 98%, 99%, or 100%. Each possibility represents a separate embodiment of the present invention. In one embodiment, the at least one nucleotide which differs between the mutated allele and the functional allele, is a disease-associated mutation. In some embodiments, the SNP is highly prevalent in the population. In such embodiments, “highly prevalent” refers to at least 10%, 11%, 12%, 13%, 14%, 15%, 20%, 30%, 40%, 50%, 60%, or 70% of a population. Each possibility represents a separate embodiment of the present invention.

### Delivery to Cells

The compositions described herein may be delivered to a target cell by any suitable means. Compositions of the present invention may be targeted to any cell which contains and/or expresses a mutated allele, including any mammalian cell, for example a retinal pigment epithelium (RPE) cell. For example, in one embodiment an RNA molecule of the present invention that specifically targets a mutated RPE65 allele is delivered to a target cell and the target cell is a stem cell or a retinal pigment epithelium cell. The delivery to the cell may be performed in-vitro, ex-vivo, or in-vivo. Further, the compositions described herein may comprise any one or more of a DNA molecule, an RNA molecule, a ribonucleoprotein (RNP), a nucleic acid vector, or any combination thereof. In some embodiments, the composition is a naked DNA plasmid. In some embodiments, the composition is a naked RNA. In some embodiments, the composition is an RNP. An RNP composition may be conjugated to a cell-penetrating peptide (CPP), an antibody, a targeting moiety, or any combination thereof.

In some embodiments, the composition is packaged into an adeno-associated virus (AAV). In some embodiments, the composition is packaged into a lentivirus, such as a non-integrating lentivirus or a lentivirus lacking reverse transcription capability. In some embodiments, the composition is packaged into liposomes, extracellular vesicles, or exosomes, which may be pseudotyped with vesicular stomatitis glycoprotein (VSVG) or conjugated to a cell-penetrating peptide, an antibody, a targeting moiety, or any combination thereof.

In preferred embodiments, the composition is delivered in-vivo to retinal pigment epithelium cells within the eye of a subject. The in-vivo delivery to an eye of a subject my occur by subretinal injection, suprachoroidal injection, or injection to the interior chamber of the eye. The injected composition may be packaged in adeno-associated virus (AAV), lentivirus, preferably a non-integrating lentivirus, liposomes, extracellular vesicles, or exosomes. In some embodiments, the injected exosome may be pseudotyped with vesicular stomatitis glycoprotein (VSVG) or conjugated to a cell-penetrating peptide, an antibody, a targeting moiety, or any combination thereof.

In other embodiments, the composition is delivered to a cell ex-vivo. In some embodiments, the cell is a stem cell. In some embodiments, the stem cell is an autologous pluripotent stem cell or an induced pluripotent stem cell (iPSC). In some embodiments, the stem cell is differentiated into a retinal pigment epithelium cell. In some embodiments, the cell is a retinal pigment epithelium cell. The composition may be delivered to the cell by any known ex-vivo delivery method, including but not limited to, electroporation, viral transduction, nanoparticle delivery, liposomes, exosomes etc. Upon ex-vivo delivery of the composition to a cell, the cell may be introduced into the eye of a subject. In one example, the composition is delivered ex-vivo to iPSCs or IPSC-derived retinal pigment epithelium cells expanded into a patch or a tissue that is to be surgically reintroduced to the eye (See Sharma et al. 2019). Additional detailed delivery methods are described throughout this section.

In some embodiments, the RNA molecule comprises a chemical modification. Non-limiting examples of suitable chemical modifications include 2′-O-methyl (M). 2′-O-methyl. 3′phosphorothioate (MS) or 2′-O-methyl, 3′thioPACE (MSP), pseudouridine, and 1-methyl pseudo-uridine. Each possibility represents a separate embodiment of the present invention.

Any suitable viral vector system may be used to deliver nucleic acid compositions e.g., the compositions of the subject invention. Conventional viral and non-viral based gene transfer methods can be used to introduce nucleic acids and target tissues. In certain embodiments, nucleic acids are administered for in vivo or ex vivo gene therapy uses. Non-viral vector delivery systems include naked nucleic acid, and nucleic acid complexed with a delivery vehicle such as a liposome or poloxamer. For a review of gene therapy procedures, see Anderson (1992); Nabel & Felgner (1993); Mitani & Caskey (1993); Dillon (1993); Miller (1992); Van Brunt (1988); Vigne (1995); Kremer & Perricaudet (1995); Haddada et al. (1995); and Yu et al. (1994).

Methods of non-viral delivery of nucleic acids and/or proteins include electroporation, lipofection, microinjection, biolistics, particle gun acceleration, virosomes, liposomes, immunoliposomes, lipid nanoparticles (LNPs), polycation or lipid:nucleic acid conjugates, artificial virions, and agent-enhanced uptake of nucleic acids or can be delivered to plant cells by bacteria or viruses (e.g., Agrobacterium, Rhizobium sp. NGR234, Sinorhizoboiummeliloti, Mesorhizobium loti, tobacco mosaic virus, potato virus X, cauliflower mosaic virus and cassava vein mosaic virus). (See, e.g., Chung et al., 2006). Sonoporation using, e.g., the Sonitron 2000 system (Rich-Mar), can also be used for delivery of nucleic acids. Cationic-lipid mediated delivery of proteins and/or nucleic acids is also contemplated as an in vivo, ex vivo, or in vitro delivery method. (See Zuris et al. (2015); see also Coelho et al. (2013); Judge et al. (2006); and Basha et al. (2011)).

Additional exemplary nucleic acid delivery systems include those provided by Amaxa® Biosystems (Cologne, Germany), Maxcyte, Inc. (Rockville, Md.), BTX Molecular Delivery Systems (Holliston, Mass.) and Copernicus Therapeutics Inc., (see, e.g., U.S. Pat. No. 6,008,336). Lipofection is described in e.g., U.S. Pat. Nos. 5,049,386, 4,946,787; and 4,897,355, and lipofection reagents are sold commercially (e.g., Transfectam™, Lipofectin™ and Lipofectamine™ RNAiMAX). Cationic and neutral lipids that are suitable for efficient receptor-recognition lipofection of polynucleotides include those disclosed in PCT International Publication Nos. WO/1991/017424 and WO/1991/016024. Delivery can be to cells (ex vivo administration) or target tissues (in vivo administration).

The preparation of lipid:nucleic acid complexes, including targeted liposomes such as immunolipid complexes, is well known to one of skill in the art (see, e.g., Crystal, Science (1995); Blaese et al., (1995); Behr et al., (1994); Remy et al. (1994); Gao and Huang (1995); Ahmad and Allen (1992); U.S. Pat. Nos. 4,186,183; 4,217,344; 4,235,871; 4,261,975; 4,485,054; 4,501,728; 4,774,085; 4,837,028; and 4,946,787).

Additional methods of delivery include the use of packaging the nucleic acids to be delivered into EnGeneIC delivery vehicles (EDVs). These EDVs are specifically delivered to target tissues using bispecific antibodies where one arm of the antibody has specificity for the target tissue and the other has specificity for the EDV. The antibody brings the EDVs to the target cell surface and then the EDV is brought into the cell by endocytosis. Once in the cell, the contents are released (See MacDiarmid et al., 2009).

The use of RNA or DNA viral based systems for viral mediated delivery of nucleic acids take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of nucleic acids include, but are not limited to, retroviral, lentivirus, adenoviral, adeno-associated, vaccinia and herpes simplex virus vectors for gene transfer.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system depends on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immunodeficiency virus (SIV), human immunodeficiency virus (HIV), and combinations thereof (See, e.g., Buchschacher et al. (1992); Johann et al. (1992); Sommerfelt et al. (1990); Wilson et al. (1989); Miller et al. (1991); PCT International Publication No. WO/1994/026877A1).

At least six viral vector approaches are currently available for gene transfer in clinical trials, which utilize approaches that involve complementation of defective vectors by genes inserted into helper cell lines to generate the transducing agent.

pLASN and MFG-S are examples of retroviral vectors that have been used in clinical trials (See Dunbar et al., 1995; Kohn et al., 1995; Malech et al., 1997). PA317/pLASN was the first therapeutic vector used in a gene therapy trial (Blaese et al., 1995). Transduction efficiencies of 50% or greater have been observed for MFG-S packaged vectors. (Ellem et al., (1997); Dranoff et al., 1997).

Packaging cells are used to form virus particles that are capable of infecting a host cell. Such cells include 293 cells, which package adenovirus, AAV, and Psi-2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additionally, AAV can be produced at clinical scale using baculovirus systems (see U.S. Pat. No. 7,479,554).

In many gene therapy applications, it is desirable that the gene therapy vector be delivered with a high degree of specificity to a particular tissue type. Accordingly, a viral vector can be modified to have specificity for a given cell type by expressing a ligand as a fusion protein with a viral coat protein on the outer surface of the virus. The ligand is chosen to have affinity for a receptor known to be present on the cell type of interest. For example, Han et al. (1995) reported that Moloney murine leukemia virus can be modified to express human heregulin fused to gp70, and the recombinant virus infects certain human breast cancer cells expressing human epidermal growth factor receptor. This principle can be extended to other virus-target cell pairs, in which the target cell expresses a receptor and the virus expresses a fusion protein comprising a ligand for the cell-surface receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences which favor uptake by specific target cells.

Gene therapy vectors can be delivered in vivo by administration to an individual patient, typically by systemic administration (e.g., intravitreal, intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, optionally after selection for cells which have incorporated the vector. A non-limiting exemplary ex vivo approach may involve removal of tissue (e.g., peripheral blood, bone marrow, and spleen) from a patient for culture, nucleic acid transfer to the cultured cells (e.g., hematopoietic stem cells), followed by grafting the cells to a target tissue (e.g., bone marrow, and spleen) of the patient. In some embodiments, the stem cell or hematopoietic stem cell may be further treated with a viability enhancer.

Ex-vivo cell transfection for diagnostics, research, or for gene therapy (e.g., via re-infusion of the transfected cells into the host organism) is well known to those of skill in the art. In a preferred embodiment, cells are isolated from the subject organism, transfected with a nucleic acid composition, and re-infused back into the subject organism (e.g., patient). Various cell types suitable for ex vivo transfection are well known to those of skill in the art (See, e.g., Freshney, “Culture of Animal Cells, A Manual of Basic Technique and Specialized Applications (6th edition, 2010) and the references cited therein for a discussion of how to isolate and culture cells from patients).

Suitable cells include, but are not limited to, eukaryotic cells and/or cell lines. Non-limiting examples of such cells or cell lines generated from such cells include COS, CHO (e.g., CHO—S, CHO-K1, CHO-DG44, CHO-DUXB11, CHO-DUKX, CHOKISV), VERO, MDCK, WI38, V79, B14AF28-G3, BHK, HaK, NSO, SP2/0-Ag14, HeLa, HEK293 (e.g., HEK293-F, HEK293-H, HEK293-T), perC6 cells, any plant cell (differentiated or undifferentiated), as well as insect cells such as Spodopterafugiperda (Sf), or fungal cells such as Saccharomyces, Pichia and Schizosaccharomyces. In certain embodiments, the cell line is a CHO-K1, MDCK or HEK293 cell line. Additionally, primary cells may be isolated and used ex vivo for reintroduction into the subject to be treated following treatment with a guided nuclease system (e.g. CRISPR/Cas). Suitable primary cells include peripheral blood mononuclear cells (PBMC), and other blood cell subsets such as, but not limited to, CD4+ T cells or CD8+ T cells. Suitable cells also include stem cells such as, by way of example, embryonic stem cells, induced pluripotent stem cells, hematopoietic stem cells (CD34+), neuronal stem cells and mesenchymal stem cells.

In one embodiment, stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating CD34+ cells in vitro into clinically important immune cell types using cytokines such a GM-CSF, IFN-gamma, and TNF-alpha are known (as a non-limiting example see, Inaba et al., 1992).

Stem cells are isolated for transduction and differentiation using known methods. For example, stem cells are isolated from bone marrow cells by panning the bone marrow cells with antibodies which bind unwanted cells, such as CD4+ and CD8+ (T cells), CD45+ (panB cells), GR-1 (granulocytes), and lad (differentiated antigen presenting cells) (as a non-limiting example, see Inaba et al., 1992). Stem cells that have been modified may also be used in some embodiments.

Vectors (e.g., retroviruses, liposomes, etc.) containing therapeutic nucleic acid compositions can also be administered directly to an organism for transduction of cells in vivo. Administration is by any of the routes normally used for introducing a molecule into ultimate contact with blood or tissue cells including, but not limited to, injection, infusion, topical application (e.g., eye drops and cream) and electroporation. Suitable methods of administering such nucleic acids are available and well known to those of skill in the art, and, although more than one route can be used to administer a particular composition, a particular route can often provide a more immediate and more effective reaction than another route. According to some embodiments, the composition is delivered via IV injection.

Vectors suitable for introduction of transgenes into immune cells (e.g., T-cells) include non-integrating lentivirus vectors. See, e.g., U.S. Patent Publication No. 2009/0117617.

Pharmaceutically acceptable carriers are determined in part by the particular composition being administered, as well as by the particular method used to administer the composition. Accordingly, there is a wide variety of suitable formulations of pharmaceutical compositions available, as described below (See, e.g., Remington's Pharmaceutical Sciences, 17th ed., 1989).

In accordance with some embodiments, there is provided an RNA molecule which binds to/associates with and/or directs the RNA guided DNA nuclease to a sequence comprising at least one nucleotide which differs between a mutated allele and a functional allele (e.g., SNP) of a gene of interest (i.e., a sequence of the mutated allele which is not present in the functional allele). The sequence may be within the disease associated mutation. The sequence may be upstream or downstream to the disease associated mutation. Any sequence difference between the mutated allele and the functional allele may be targeted by an RNA molecule of the present invention to inactivate the mutant allele, or otherwise disable its dominant disease-causing effects, while preserving the activity of the functional allele.

The disclosed compositions and methods may also be used in the manufacture of a medicament for treating dominant genetic disorders in a patient.

### Mechanisms of Action for RPE65 Knockout Methods

Without being bound by any theory or mechanism, the instant invention may be utilized to apply a CRISPR nuclease to process a mutated pathogenic RPE65 allele and not a functional RPE65 allele, such as to prevent expression of the mutated pathogenic allele or to produce a truncated non-pathogenic peptide from the mutated pathogenic allele, in order to prevent or treat a dominant RPE65 gene disorder. A specific guide sequence may be selected from Table 1 based on the targeted SNP position and the type of CRISPR nuclease used (e.g. according to a required PAM sequence).

The RPE65 gene is located in chromosome 1 and encodes the retinal pigment epithelium-specific 65 kDa protein. Editing strategies for RPE65 include (1) truncation strategies requiring only one guide; (2) truncation strategies using two guides; (3) knockout strategies using two guides; and (4) two guide strategies using a first RNA guide specifically targeting a pathogenic mutation in Exon 13 (i.e. one which leads to Asp477Gly) and a second, non-discriminatory RNA guide.

An example of a truncation strategy requiring only one guide RNA molecule includes targeting a pathogenic mutation in order to mediate truncation or nonsense mediated decay (NMD) of an RPE65 mutant allele. As a non-limiting example, a frameshift in a mutated RPE65 allele may be introduced by utilizing one RNA molecule to target a pathogenic mutation in a coding exon of the mutated RPE65 allele in order to mediate a double-strand break, which leads to generation of a frameshift mutation and expression of a truncated protein or nonsense mediated decay (NMD) of its transcripts.

An example of a truncation strategy using two guides includes excision of any one of Exons 5, 6, 9, or 10 by targeting RNA molecules to flanking regions of the exons. One of the two guides must specifically target a mutated RPE65 allele over a functional RPE65 allele, for example, by targeting a SNP position.

Examples of a knockout strategy using two guides include multiple approaches. In one approach, knockout of an RPE65 mutant allele may be achieved by excision of Exon 1 (including the 5′UTR and ORF). Exon 1 may be excised by utilizing SNP positions in Intron 1 or upstream to the promoter region. Only one of the two guides needs to be a discriminatory guide. For example, Exon 1 may be excised by targeting a first RNA molecule to a SNP position in Intron 1 and a second, non-discriminatory RNA molecule targeting a region upstream to the promoter region. Alternatively, Exon 1 may be excised by targeting a first RNA molecule to a SNP position in a region upstream to the promoter region and a second, non-discriminatory RNA molecule targeting Intron 1.

In another approach using two guides, knockout of an RPE65 mutant may be achieved by excision of Exon 2. Exon 2 may be excised by utilizing SNP positions in Intron 1 or Intron 2, however only one of the two guides needs to be a discriminatory guide. Exon 2 excision in this manner generates a peptide of only 23 amino acids.

In yet another approach using two guides, knockout of an RPE65 mutant may be achieved by excision of Exon 14. Exon 14 may be excised by utilizing a SNP position downstream of Exon 14 or in Intron 13. However only one of the two guides needs to be a discriminatory guide. Exon 14 excision in this manner would eliminate the 3′UTR and thereby destabilize the transcript.

Examples of two-guide strategies using a first RNA guide specifically targeting a pathogenic mutation in Exon 13 of RPE65 (i.e. one which leads to Asp477Gly) and a second, non-discriminatory RNA guide include multiple approaches.

For example, in one approach excision from Exon 11 to a pathogenic mutation in Exon 13 is carried out. To facilitate the excision, an allele specific cut is mediated by targeting a first RNA molecule to a pathogenic mutation in Exon 13, and a biallelic cut is mediated by targeting a second, non-discriminatory RNA molecule to Intron 10. Intron 10 is preferably targeted since Intron 11 and Intron 12 are very short and therefore targeting them might cause legions that would be deleterious to a functional RPE65 allele, as well.

In another approach, excision from a pathogenic mutation in Exon 13 to Intron 13 is carried out. To facilitate the excision, an allele specific cut is mediated by targeting a first RNA molecule to a pathogenic mutation in Exon 13, and a biallelic cut is mediated by targeting a second, non-discriminatory RNA molecule to Intron 13. This approach would lead to the elimination of the splice donor at the end of Exon 13. This approach would lead to nonsense-mediated decay (NMD) or to the extension of Exon 13, which would contain stop codons and result in expression of a truncated protein.

In yet another approach, excision from a pathogenic mutation in Exon 13 to the 3′UTR is carried out. To facilitate the excision, an allele specific cut is mediated by targeting a first RNA molecule to a pathogenic mutation in Exon 13, and a biallelic cut is mediated by targeting a second, non-discriminatory RNA downstream to the 3′UTR in Exon 14. An excision performed using this approach would destabilize the transcript of the mutant RPE65 allele.

### Examples of RNA Guide Sequences which Specifically Target Mutated Alleles of RPE65 Gene

Although a large number of guide sequences can be designed to target a mutated allele, the nucleotide sequences described in Table 1 identified by SEQ ID NOs: 1-49516 below were specifically selected to effectively implement the methods set forth herein and to effectively discriminate between alleles.

Table 1 shows guide sequences designed for use as described in the embodiments above to associate with different SNPs or pathogenic mutations within a sequence of a mutated RPE65 allele. Each engineered guide molecule is further designed such as to associate with a target genomic DNA sequence of interest that lies next to a protospacer adjacent motif (PAM), e.g., a PAM matching the sequence NGG or NAG, where “N” is any nucleobase. The guide sequences were designed to work in conjunction with one or more different CRISPR nucleases, including, but not limited to, e.g. SpCas9WT (PAM SEQ: NGG), SpCas9.VQR.1 (PAM SEQ: NGAN), SpCas9.VQR2 (PAM SEQ: NGNG), SpCas9.EQR (PAM SEQ: NGAG), SpCas9.VRER (PAM SEQ: NGCG), SaCas9WT (PAM SEQ: NNGRRT), NmCas9WT (PAM SEQ: NNNNGATT), Cpf1 (PAM SEQ: TITV), or JeCas9WT (PAM SEQ: NNNVRYM). RNA molecules of the present invention are each designed to form complexes in conjunction with one or more different CRISPR nucleases and designed to target polynucleotide sequences of interest utilizing one or more different PAM sequences respective to the CRISPR nuclease utilized.

TABLE 1
Guide sequences designed to associate with specific RPE65 gene targets

| | SEQ ID NOs: | SEQ ID NOs: | SEQ ID NOs: |
|---|---|---|---|
| | of 20 base | of 21 base | of 22 base |
| Target | guides | guides | guides |
| 1:68431085_T_C | 1-46 | 47-94 | 95-144 |
| 1:68446825_G_A | 145-190 | 191-238 | 239-288 |
| 1:68425933_C_CAT | 289-302 | 303-311 | 312-324 |
| rs60701104_REF | | | |
| 1:68426291_T_C | | 325-326 | 327-330 |
| rs9436400_SNP | | | |
| 1:68426379_TA_T | 331-347 | 348-364 | 365-381 |
| rs868541802_REF | | | |
| 1:68426406_C_T | 382-389 | 390-393 | 394-399 |
| rs3125890_REF | | | |
| 1:68426406_C_T | 400-401 | | |
| rs3125890_SNP | | | |
| 1:68426632_A_G | 402-414 | 415-432 | 433-456 |
| rs75159457_REF | | | |
| 1:68426632_A_G | 403 405 407 | 417-418 420 | 435 437-438 |
| rs75159457_SNP | 411 457-488 | 428-429 489- | 441 452-453 |
| | | 523 | 524-561 |
| 1:68427338_GT_G | 562-574 | 575-587 | 588-602 |
| rs1205919238_REF | | | |
| 1:68427338_GT_G | 603 | | 604 |
| rs1205919238_SNP | | | |
| 1:68427665_T_A | 605-608 | 609-612 | 613-619 |
| rs11209300_REF | | | |
| 1:68427665_T_A | 605-608 | 609-612 | 613-618 620 |
| rs11209300_SNP | | | |
| 1:68428071_G_A | 621-666 | 667-714 | 715-764 |
| rs4264030_REF | | | |
| 1:68428071_G_A | 627 632 636 | 673 678 682 | 721 726 730 |
| rs4264030_SNP | 640 648 653 | 686 695 703 | 732 735 737 |
| | 656 662 765- | 709 711 800- | 753 761 831- |
| | 799 | 830 | 870 |
| 1:68428149_T_C | 871-914 | 915-957 | 958-1003 |
| rs2419988_REF | | | |
| 1:68428149_T_C | 875 885 891- | 919 921 935 | 964 973 979- |
| rs2419988_SNP | 892 894 914 | 938 1042-1081 | 980 983 1082- |
| | 1004-1041 | | 1123 |
| 1:68428613_T_C | 1124-1132 | 1133-1134 | 1135-1140 |
| rs3118415_REF | | | |
| 1:68428613_T_C | 1124-1125 1127 | 1133 1160-1170 | 1139 1171-1185 |
| rs3118415_SNP | 1130 1141-1159 | | |
| 1:68428861_G_C | 1186-1208 | 1209-1216 | 1217-1226 |
| rs3118416_REF | | | |
| 1:68428861_G_C | 1227-1249 | 1250-1257 | 1258-1267 |
| rs3118416_SNP | | | |
| 1:68429047_G_GCT | 1268-1279 | | |
| rs149739986_SNP | | | |
| 1:68429165_C_T | 1280-1294 | 1295-1307 | 1308-1322 |
| rs2182315_REF | | | |
| 1:68429165_C_T | 1281-1282 | 1295 1301-1302 | 1309 1315 1320 |
| rs2182315_SNP | 1288-1289 | 1306 1330-1336 | 1322 1337-1345 |
| | 1323-1329 | | |
| 1:68429245_T_C | 1346-1372 | 1373-1393 | 1394-1418 |
| rs3118418_REF | | | |
| 1:68429245_T_C | 1358 1361 1364 | 1382 1385 | 1408 1415 |
| rs3118418_SNP | 1370 1419-1447 | 1392-1393 | 1417-1418 |
| | | 1448-1472 | 1473-1501 |
| 1:68430961_G_A | 1502-1529 | 1530-1543 | 1544-1561 |
| rs932783_REF | | | |
| 1:68430961_G_A | 1517 1562-1571 | 1572-1575 | 1576-1581 |
| rs932783_SNP | | | |
| 1:68432081_G_A | 1582-1600 | 1601-1619 | 1620-1638 |
| rs12124063_REF | | | |
| 1:68432081_G_A | 1592 1595 | 1611 1613 1615 | 1628 1630 1634 |
| rs12124063_SNP | 1599-1600 | 1618 1654-1668 | 1637 1669-1683 |
| | 1639-1653 | | |
| 1:68432262_C_G | 1684-1729 | 1730-1777 | 1778-1827 |
| rs77585943_REF | | | |
| 1:68432262_C_G | 1685 1687 | 1731 1733 1736 | 1781 1784 1792 |
| rs77585943_SNP | 1695-1696 1703 | 1743 1750 1755 | 1797 1800 1805 |
| | 1708 1712 1725 | 1759 1765 | 1809 1815 |
| | 1828-1865 | 1866-1905 | 1906-1947 |
| 1:68433374_G_A | 1948-1993 | 1994-2041 | 2042-2091 |
| rs1886906_REF | | | |
| 1:68433374_G_A | 1956-1957 1971 | 2002-2003 2010 | 2048 2051-2052 |
| rs1886906_SNP | 1975 1977 1980 | 2019 2023 2025 | 2059 2068 2074 |
| | 1982 1992 | 2030 2040 | 2079 2082 |
| | 2092-2129 | 2130-2169 | 2170-2211 |
| 1:68433703_A_G | 2212-2248 | 2249-2275 | 2276-2308 |
| rs3125891_REF | | | |
| 1:68433703_A_G | 2217-2218 2225 | 2251 2256 2264 | 2276 2280 |
| rs3125891_SNP | 2236 2243 2245 | 2271 2274-2275 | 2284-2285 2304 |
| | 2248 2309-2346 | 2347-2382 | 2307-2308 |
| | | | 2383-2424 |
| 1:68433977_G_A | 2425-2470 | 2471-2518 | 2519-2568 |
| rs11581095_REF | | | |
| 1:68433977_G_A | 2426 2431-2432 | 2472 2474 | 2519 2521 2523 |
| rs11581095_SNP | 2435 2437-2438 | 2478-2479 2482 | 2527 2531 2534 |
| | 2457 2464 | 2485 2504-2505 | 2553 2563 |
| | 2569-2606 | 2607-2646 | 2647-2688 |
| 1:68434079_G_A | 2689-2734 | 2735-2781 | 2782-2829 |
| rs12030710_REF | | | |
| 1:68434079_G_A | 2694 2696-2697 | 2740 2742-2743 | 2787 2789-2790 |
| rs12030710_SNP | 2717 2720 | 2764 2767 | 2814 2816-2817 |
| | 2722-2723 2731 | 2769-2770 | 2908-2949 |
| | 2830-2867 | 2868-2907 | |
| 1:68434091_GAT_G | 2689 2691 2693 | 2735 2737 2739 | 2782 2784 2786 |
| rs1003041423_REF | 2696-2700 2702 | 2741 2743-2747 | 2788 2790-2794 |
| | 2708 2712 2714 | 2749 2755 2759 | 2796 2802 2806 |
| | 2716 2718 | 2761 2763 2765 | 2808 2810 2812 |
| | 2722-2726 2728 | 2769-2773 2775 | 2815 2817-2821 |
| | 2730 2733 | 2777 2780 | 2823 2825 2828 |
| | 2950-2954 | 2955-2958 | 2959-2961 |
| 1:68434556_T_C | 2962-3007 | 3008-3055 | 3056-3105 |
| rs3118419_REF | | | |
| 1:68434556_T_C | 2962-2964 | 3008 3010 | 3056 3061 3065 |
| rs3118419_SNP | 2970-2971 2981 | 3016-3017 3032 | 3081 3084 |
| | 2986 2991 | 3035 3038-3039 | 3087-3088 3101 |
| | 3106-3143 | 3144-3183 | 3184-3225 |
| 1:68434669_A_AT | 3226-3236 | | |
| rs5774935_REF | | | |
| 1:68434669_A_AT | 3232 3234 | | |
| rs5774935_SNP | | | |
| 1:68434719_C_CTGTT | 3237-3246 | 3247 | 3248-3251 |
| rs150459448_REF | | | |
| 1:68434729_G_T | 3237-3243 | 3247 | 3248-3251 |
| rs1555845_REF | 3245-3246 | | |
| 1:68434877_C_T | | 3252-3253 | 3254-3260 |
| rs1555846_REF | | | |
| 1:68434877_C_T | 3261-3270 | 3271-3282 | 3283-3298 |
| rs1555846_SNP | | | |
| 1:68435174_GCTCTTGTTTC- | 3299-3344 | 3345-3392 | 3393-3442 |
| TTTTTCTGGCTT_G | | | |
| rs11269074_REF | | | |
| 1:68435749_T_G | 3443-3483 | 3484-3524 | 3525-3567 |
| rs3790469_REF | | | |
| 1:68435749_T_G | 3449 3459 3469 | 3490 3500 3505 | 3530 3542 3547 |
| rs3790469_SNP | 3474 3568-3605 | 3515 3606-3644 | 3558 3645-3685 |
| 1:68435988_T_C | 3686-3708 | 3709-3731 | 3732-3756 |
| rs3125894_REF | | | |
| 1:68435988_T_C | 3686 3688 3692 | 3709 3711 3714 | 3732 3734 3739 |
| rs3125894_SNP | 3698 3757-3787 | 3722 3788-3808 | 3747 3809-3831 |
| 1:68436052_C_A | 3832-3875 | 3876-3918 | 3919-3963 |
| rs3125895_REF | | | |
| 1:68436052_C_A | 3840 3864 3866 | 3884 3915 3917 | 3921 3928 3962 |
| rs3125895_SNP | 3871 3873-3874 | 4002-4041 | 4042-4083 |
| | 3964-4001 | | |
| 1:68436153_G_A | 4084-4097 | | 4098-4099 |
| rs3125896_REF | | | |
| 1:68436153_G_A | 4085 4090 | | |
| rs3125896_SNP | | | |
| 1:68436172_G_A | 4085 4090 | 4132-4151 | 4099 4152-4175 |
| rs3125897_REF | 4100-4131 | | |
| 1:68436172_G_A | 4176-4191 | 4192-4199 | 4200-4211 |
| rs3125897_SNP | | | |
| 1:68436228_T_C | 4212-4257 | 4258-4305 | 4306-4355 |
| rs3125898_REF | | | |
| 1:68436228_T_C | 4218-4219 4222 | 4264-4265 4268 | 4312 4314 4320 |
| rs3125898_SNP | 4227-4228 4243 | 4271 4274-4275 | 4323-4324 4330 |
| | 4245 4256 | 4290 4293 | 4340 4343 |
| | 4356-4393 | 4394-4433 | 4434-4475 |
| 1:68436309_T_C | 4476-4521 | 4522-4569 | 4570-4619 |
| rs17130688_REF | | | |
| 1:68436309_T_C | 4482 4485 4490 | 4526 4535 4538 | 4574 4583 4588 |
| rs17130688_SNP | 4493 4495 4505 | 4541 4543 4553 | 4590 4592 |
| | 4510 4520 | 4558 4568 | 4607-4608 4618 |
| | 4620-4657 | 4658-4697 | 4698-4739 |
| 1:68436444_CTATTTATT_C | 4740-4758 | 4759-4777 | 4778-4798 |
| rs938759267_REF | | | |
| 1:68436444_CTATT_C | 4740-4758 | 4759-4777 | 4778-4798 |
| rs938759267_REF | | | |
| 1:68436702_G_A | 4799-4831 | 4832-4866 | 4867-4903 |
| rs34194247_REF | | | |
| 1:68436715_G_A | 4799 4801 | 4832 4834 | 4867 4869-4873 |
| rs3125900_REF | 4803-4805 | 4836-4838 | 4875-4880 |
| | 4807-4812 4815 | 4840-4845 | 4882-4883 |
| | 4817-4818 | 4847-4848 | 4886-4887 |
| | 4820-4823 | 4851-4852 | 4889-4894 |
| | 4827-4828 4904 | 4854-4858 | 4898-4899 4906 |
| | | 4862-4863 4905 | |
| 1:68436715_G_A | 4805 4807-4808 | 4840-4841 4847 | 4870 4876 4882 |
| rs3125900_SNP | 4827 4907-4927 | 4862 4928-4952 | 4898 4953-4981 |
| 1:68436720_G_A | 4801 4803 | 4832 4834 4836 | 4867 4869 4871 |
| rs79716012_REF | 4809-4810 4812 | 4842-4843 4845 | 4873 4877-4878 |
| | 4815 4817 4820 | 4848 4851 4854 | 4880 4883 4886 |
| | 4822 4828 4904 | 4856-4857 4863 | 4889 4891-4892 |
| | | 4905 | 4894 4899 4906 |
| 1:68436720_G_A | 4801 4810 4815 | 4832 4834 4843 | 4867 4869 4873 |
| rs79716012_SNP | 4828 4982-4992 | 4848 4993-5007 | 4878 5008-5026 |
| 1:68437256_C_G | 5027-5072 | 5073-5119 | 5120-5169 |
| rs75711879_REF | | | |
| 1:68437256_C_G | 5037 5040 5042 | 5086 5088 5095 | 5135 5143 |
| rs75711879_SNP | 5049 5051-5052 | 5097-5098 | 5145-5146 5151 |
| | 5064-5065 | 5111-5112 | 5160-5161 5165 |
| | 5170-5207 | 5208-5247 | 5248-5289 |
| 1:68438259_C_T | 5290-5312 | 5313-5335 | 5336-5358 |
| rs12145904_REF | | | |
| 1:68438259_C_T | 5291 5296 5306 | 5314 5318-5319 | 5336 5340-5341 |
| rs12145904_SNP | 5308 5359-5377 | 5331 5378-5396 | 5343 5397-5415 |
| 1:68438583_T_C | 5416-5461 | 5462-5509 | 5510-5559 |
| rs3125902_REF | | | |
| 1:68438583_T_C | 5416 5420 5423 | 5462 5466 5469 | 5510 5517 5525 |
| rs3125902_SNP | 5429-5431 | 5477 5479 | 5527 5532-5533 |
| | 5436-5437 | 5483-5485 | 5541 5549 |
| | 5560-5597 | 5598-5637 | 5638-5679 |
| 1:68438672_G_T | 5680-5722 | 5723-5766 | 5767-5814 |
| rs3118420_REF | | | |
| 1:68438672_G_T | 5688 5693 5704 | 5726 5732 5737 | 5771 5777 5779 |
| rs3118420_SNP | 5710 5718 | 5748 5754 | 5794 5796 5802 |
| | 5815-5849 | 5850-5882 | 5883-5919 |
| 1:68438830_A_G | 5920-5964 | 5965-5999 | 6000-6041 |
| rs12138573_REF | | | |
| 1:68438830_A_G | 5926 5932 5934 | 5971 5977 5980 | 6006 6012 6016 |
| rs12138573_SNP | 5937 5939 5942 | 5982 5992 | 6018 6023 |
| | 5944 6042-6079 | 6080-6119 | 6033-6034 |
| | | | 6120-6161 |
| 1:68439675_G_C | 6162-6196 | 6197-6231 | 6232-6266 |
| rs1925955_REF | | | |
| 1:68439675_G_C | 6174 6184-6185 | 6205 6210 6220 | 6237 6241 6246 |
| rs1925955_SNP | 6191 6267-6297 | 6226 6298-6328 | 6256 6329-6359 |
| 1:68440298_T_A | 6360-6380 | 6381-6399 | 6400-6422 |
| rs17130691_REF | | | |
| 1:68440298_T_A | 6367 6374 6377 | 6386 6390 6396 | 6407 6411 6418 |
| rs17130691_SNP | 6380 6423-6439 | 6399 6440-6454 | 6422 6455-6473 |
| 1:68440407_G_A | 6474-6499 | 6500-6503 | 6504-6514 |
| rs3125904_REF | | | |
| 1:68440407_G_A | 6515-6516 | | 6507 |
| rs3125904_SNP | | | |
| 1:68441530_T_C | 6517-6552 | 6553-6583 | 6584-6616 |
| rs78507000_REF | | | |
| 1:68441530_T_C | 6519 6522 6545 | 6555 6577 | 6609 6614 |
| rs78507000_SNP | 6617-6651 | 6652-6686 | 6687-6723 |
| 1:68441814_GA_G | 6724-6732 | | |
| rs150774295_REF | | | |
| 1:68441814_GA_G | 6727 6733-6740 | | |
| rs150774295_SNP | | | |
| 1:68441844_A_C | 6741-6758 | 6759-6775 | 6776-6794 |
| rs3125905_REF | | | |
| 1:68441844_A_C | 6741-6742 6756 | 6759 6773 | 6791 6794 |
| rs3125905_SNP | 6795-6814 | 6815-6831 | 6832-6854 |
| 1:68442800_G_A | 6855-6900 | 6901-6948 | 6949-6998 |
| rs2038900_REF | | | |
| 1:68442800_G_A | 6859 6862 6866 | 6902 6906 6909 | 6950 6954 6957 |
| rs2038900_SNP | 6868-6869 6873 | 6915-6917 6921 | 6963 6965 6972 |
| | 6876 6900 | 6924 7037-7075 | 6981 6985 |
| | 6999-7036 | | 7076-7117 |
| 1:68442818_T_C | 6856 6859-6860 | 6901-6903 | 6949-6951 |
| rs2038901_REF | 6863 6866 | 6906-6907 6910 | 6954-6955 6958 |
| | 6868-6869 6874 | 6913 6916-6917 | 6961 6964-6965 |
| | 6877 6887 | 6922 6925 6935 | 6970 6973 |
| | 7118-7153 | 7154-7189 | 6980-6981 6984 |
| | | | 7190-7225 |
| 1:68442818_T_C | 6856 6863 6874 | 6901 6903 6910 | 6949 6951 6958 |
| rs2038901_SNP | 6877 7125 7143 | 6925 7160 7178 | 6980 7211 7214 |
| | 7150-7151 | 7186 7189 | 7222 7225 |
| | 7226-7263 | 7264-7303 | 7304-7345 |
| 1:68443118_CA_C | 7346-7391 | 7392-7433 | 7434-7482 |
| rs147665807_REF | | | |
| 1:68443118_CA_C | 7353 7364 7374 | 7408 7410 7420 | 7440 7442 7451 |
| rs147665807_SNP | 7377 7384 7387 | 7427 7431 | 7463 7474 7479 |
| | 7389 7483-7520 | 7521-7560 | 7561-7602 |
| 1:68443430_C_T | 7603-7648 | 7649-7696 | 7697-7746 |
| rs17130694_REF | | | |
| 1:68443430_C_T | 7604 7607 7615 | 7650 7653 7669 | 7701 7717 7720 |
| rs17130694_SNP | 7625-7626 7630 | 7672-7673 7677 | 7725 7729-7730 |
| | 7636 7639 | 7681 7684 | 7733 7742 |
| | 7747-7784 | 7785-7824 | 7825-7866 |
| 1:68443445_A_G | 7605 7607-7610 | 7651 7653-7656 | 7697 7699 |
| rs12408546_REF | 7617 7622-7623 | 7659 7663 7668 | 7701-7704 7707 |
| | 7626 7628-7631 | 7670 7673 | 7711 7716 7718 |
| | 7637 7639 7642 | 7675-7678 7681 | 7721 7723-7726 |
| | 7867-7896 | 7685 7687 7690 | 7730 7734 7736 |
| | | 7897-7926 | 7739 7742 |
| | | | 7927-7956 |
| 1:68443445_A_G | 7605 7623 7628 | 7651 7659 7670 | 7697 7699 7707 |
| rs12408546_SNP | 7637 7869 | 7685 7899 7903 | 7734 7933 7944 |
| | 7873-7874 7884 | 7914 7916 | 7946 7949 |
| | 7957-7994 | 7995-8034 | 8035-8076 |
| 1:68443820_G_A | 8077-8107 | 8108-8130 | 8131-8157 |
| rs12077372_REF | | | |
| 1:68443820_G_A | 8079 8084 8088 | 8110 8114 8117 | 8133 8140 8144 |
| rs12077372_SNP | 8102 8158-8168 | 8128 8169-8177 | 8152 8178-8192 |
| 1:68445316_C_A | 8193-8228 | 8229-8260 | 8261-8298 |
| rs3790472_REF | | | |
| 1:68445316_C_A | 8195 8197 8202 | 8229 8232 8234 | 8261-8262 8265 |
| rs3790472_SNP | 8204 8207 8209 | 8238 8243 8245 | 8267 8272 8279 |
| | 8216 8219 | 8248 8251 | 8281 8284 |
| | 8299-8314 | 8315-8325 | 8326-8341 |
| 1:68445430_A_G | 8342-8387 | 8388-8433 | 8434-8483 |
| rs3790473_REF | | | |
| 1:68445430_A_G | 8351 8353 8358 | 8391 8398 8405 | 8435 8438 8445 |
| rs3790473_SNP | 8362 8368 8372 | 8415 8419 8425 | 8460 8463 8467 |
| | 8379 8384 | 8429 8433 | 8479 8483 |
| | 8484-8521 | 8522-8561 | 8562-8603 |
| 1:68446330_T_TGCTA | 8604-8648 | 8649-8693 | 8694-8740 |
| rs147893529_REF | | | |
| 1:68446330_T_TGCTA | 8605 8612 8620 | 8657 8665 8680 | 8702 8726 8729 |
| rs147893529_SNP | 8638 8741-8771 | 8683 8772-8804 | 8737 8805-8839 |
| 1:68447072_T_C | 8840-8885 | 8886-8933 | 8934-8983 |
| rs2012235_REF | | | |
| 1:68447072_T_C | 8843 8848 8853 | 8892 8895 8897 | 8940 8945 8952 |
| rs2012235_SNP | 8859 8862 8872 | 8901 8907 8910 | 8956 8959 |
| | 8877 8879 | 8925 8927 | 8974-8975 8977 |
| | 8984-9021 | 9022-9061 | 9062-9103 |
| 1:68447254_T_G | 9104-9148 | 9149-9191 | 9192-9237 |
| rs3118423_REF | | | |
| 1:68447254_T_G | 9112 9135-9136 | 9180 9188 | 9204 9207 9234 |
| rs3118423_SNP | 9144-9145 | 9190-9191 | 9236-9237 |
| | 9147-9148 | 9276-9315 | 9316-9357 |
| | 9238-9275 | | |
| 1:68447776_T_G | 9358-9386 | 9387-9417 | 9418-9450 |
| rs2986125_REF | | | |
| 1:68447894_C_A | 9451-9467 | 9468-9484 | 9485-9501 |
| rs2986124_REF | | | |
| 1:68447894_C_A | 9456 9462-9463 | 9478-9479 | 9496 9499-9501 |
| rs2986124_SNP | 9467 9502-9512 | 9483-9484 | 9524-9534 |
| | | 9513-9523 | |
| 1:68448323_C_T | 9535-9577 | 9578-9616 | 9617-9658 |
| rs72926973_REF | | | |
| 1:68448323_C_T | 9550 9554 9562 | 9589 9593 9607 | 9629 9633 9648 |
| rs72926973_SNP | 9575 9577 | 9616 9688-9710 | 9657 9711-9741 |
| | 9659-9687 | | |
| 1:68448987_A_G | 9742-9783 | 9784-9817 | 9818-9861 |
| rs2277874_REF | | | |
| 1:68448987_A_G | 9746 9750 9752 | 9786-9787 9790 | 9822-9823 |
| rs2277874_SNP | 9758-9759 | 9792 9797-9798 | 9826-9827 |
| | 9768-9769 9778 | 9803 9806 | 9835-9836 |
| | 9862-9899 | 9900-9939 | 9844-9845 |
| | | | 9940-9981 |
| 1:68449261_A_G | 9982-9998 | 9999-10010 | 10011-10025 |
| rs3125906_REF | | | |
| 1:68449261_A_G | 9983 9985 9990 | 9999 10001 | 10011-10012 |
| rs3125906_SNP | 9996 10026- | 10005 10009 | 10016 10024 |
| | 10049 | 10050-10064 | 10065-10081 |
| 1:68449382_C_A | 10082-10126 | 10127-10168 | 10169-10216 |
| rs3118426_REF | | | |
| 1:68449382_C_A | 10103 10108 | 10151 10163 | 10187 10191 |
| rs3118426_SNP | 10111 10118 | 10165 10167 | 10196 10208 |
| | 10121 10124- | 10249-10275 | 10214-10215 |
| | 10125 10217- | | 10276-10306 |
| | 10248 | | |
| 1:68449796_A_T | 10307-10350 | 10351-10392 | 10393-10442 |
| rs3125907_REF | | | |
| 1:68449796_A_T | 10308 10313 | 10351 10353 | 10393 10395 |
| rs3125907_SNP | 10316-10317 | 10355 10359 | 10397 10401 |
| | 10322 10331 | 10362-10363 | 10405 10412 |
| | 10333 10347 | 10367 10376 | 10421 10424 |
| | 10443-10478 | 10479-10512 | 10513-10554 |
| 1:68450440_G_C | 10555-10577 | 10578-10581 | 10582-10592 |
| rs382422_REF | | | |
| 1:68450440_G_C | 10564 10570 | 10580-10581 | 10583 10586 |
| rs382422_SNP | 10572-10573 | 10612-10613 | 10589-10590 |
| | 10593-10611 | | 10614-10620 |
| 1:68450870_T_C | 10621-10661 | 10662-10702 | 10703-10745 |
| rs3118427_REF | | | |
| 1:68450870_T_C | 10622 10626 | 10663 10667- | 10704-10705 |
| rs3118427_SNP | 10641-10642 | 10668 10682 | 10709 10724 |
| | 10746-10783 | 10784-10822 | 10823-10863 |
| 1:68450986_G_A | 10864-10909 | 10910-10957 | 10958-11007 |
| rs3125908_REF | | | |
| 1:68450986_G_A | 10866 10870 | 10911 10913 | 10959-10960 |
| rs3125908_SNP | 10874-10875 | 10917 10921- | 10962 10966 |
| | 10884 10898 | 10922 10925 | 10970-10971 |
| | 10904 10908 | 10932 10946 | 10974 10999 |
| | 11008-11045 | 11046-11085 | 11086-11127 |
| 1:68451167_G_T | 11128-11156 | 11157-11181 | 11182-11208 |
| rs12759602_REF | | | |
| 1:68451167_G_T | 11132 11151 | 11169 11176 | 11195-11196 |
| rs12759602_SNP | 11155-11156 | 11180-11181 | 11207-11208 |
| | 11209-11227 | 11228-11244 | 11245-11263 |
| 1:68451599_A_G | 11264-11290 | 11291-11307 | 11308-11333 |
| rs2477974_REF | | | |
| 1:68451599_A_G | 11266 11272 | 11293 11297 | 11312 11314 |
| rs2477974_SNP | 11281 11288 | 11300 11306 | 11318 11322 |
| | 11334-11371 | 11372-11402 | 11325 11403- |
| | | | 11439 |
| 1:68451602_C_A | 11264-11265 | 11291-11292 | 11308-11311 |
| rs3125909_REF | 11267-11271 | 11294-11296 | 11313 11315- |
| | 11273-11282 | 11298-11299 | 11316 11318- |
| | 11284-11287 | 11301-11307 | 11321 11323- |
| | 11289-11290 | | 11333 |
| 1:68451602_C_A | 11278 11281 | 11302 11306 | 11318 11326 |
| rs3125909_SNP | 11440-11450 | 11451 | 11330 11452- |
| | | | 11453 |
| 1:68451671_T_C | 11454-11499 | 11500-11547 | 11548-11597 |
| rs3118428_REF | | | |
| 1:68451671_T_C | 11454-11455 | 11500 11502 | 11548 11550 |
| rs3118428_SNP | 11460-11461 | 11507-11508 | 11556 11562 |
| | 11475-11476 | 11523 11533 | 11572 11583 |
| | 11486 11497 | 11544 11547 | 11594 11597 |
| | 11598-11635 | 11636-11675 | 11676-11717 |
| 1:68452065_G_A | | | 11718-11721 |
| rs12407140_REF | | | |
| 1:68452441_G_A | 11722-11765 | 11766-11798 | 11799-11838 |
| rs72674322_REF | | | |
| 1:68452441_G_A | 11735-11736 | 11774 11778 | 11809 11813 |
| rs72674322_SNP | 11741 11757 | 11792 11797- | 11817 11823 |
| | 11761 11763- | 11798 11863- | 11836 11838 |
| | 11765 11839- | 11884 | 11885-11910 |
| | 11862 | | |
| 1:68452444_T_C | 11722-11731 | 11766-11772 | 11799-11805 |
| rs72674323_REF | 11733-11735 | 11775-11777 | 11807-11808 |
| | 11737-11746 | 11779-11784 | 11810-11812 |
| | 11748-11757 | 11786-11792 | 11814-11821 |
| | 11759-11763 | 11794-11797 | 11824-11831 |
| | 11911-11916 | 11917-11921 | 11833-11838 |
| | | | 11922-11926 |
| 1:68452444_T_C | 11728 11735 | 11771 11789 | 11817 11828 |
| rs72674323_SNP | 11749 11753 | 11795 11797 | 11834 11837- |
| | 11761 11913- | 11918-11919 | 11838 11923- |
| | 11914 11916 | 11921 11965- | 11924 11926 |
| | 11927-11964 | 12004 | 12005-12046 |
| 1:68452650_C_T | 12047-12089 | 12090-12133 | 12134-12179 |
| rs3125910_REF | | | |
| 1:68452650_C_T | 12056-12057 | 12100 12128 | 12173 12175 |
| rs3125910_SNP | 12073 12085 | 12130 12132 | 12177 12179 |
| | 12087 12180- | 12218-12256 | 12257-12297 |
| | 12217 | | |
| 1:68452696_CGT_C | 12298-12323 | 12324-12348 | 12349-12374 |
| rs1318744874_REF | | | |
| 1:68452696_C_CGT | 12298-12323 | 12324-12348 | 12349-12374 |
| rs1318744874_REF | | | |
| 1:68450655-1:68451154 | 10621-10634 | 10662-10676 | 10703-10718 |
| 700-1200 bps Upstream to | 10637-10661 | 10678-10702 | 10720-10745 |
| Transcriptional Start Site | 10864-10909 | 10910-10957 | 10958-11007 |
| | 12375-13051 | 13052-13753 | 13754-14477 |
| 1:68428322-1:68428821 | 1127-1128 | 1133-1134 | 1135 1137-1139 |
| Intergenic_500 bp Downstream to | 14478-14901 | 14902-15379 | 15380-15897 |
| Exon 14 | | | |
| 1:68437687-1:68438186 | 15898-16401 | 16402-16955 | 16956-17545 |
| Intron 10_500 bp Downstream to | | | |
| Exon 10 | | | |
| 1:68431586-1:68432085 | 17546-18205 | 18206-18885 | 18886-19585 |
| Intron 10_500 bp Upstream to | | | |
| Exon 11 | | | |
| 1:68431377-1:68431469 | 19586-19681 | 19682-19785 | 19786-19893 |
| Intron 11 | | | |
| 1:68431177-1:68431281 | 19894-19965 | 19966-20055 | 20056-20167 |
| Intron 12 | | | |
| 1:68430565-1:68431064 | 1502 1505 1510 | 1530 1532-1535 | 1544 1546 |
| Intron 13_500 bp Downstream to | 1512-1513 1516 | 1537-1538 1541 | 1549-1552 |
| Exon 13 | 1518 1521 1526 | 1543 20822- | 1554-1555 |
| | 1529 20168- | 21518 | 1558-1561 |
| | 20821 | | 21519-22246 |
| Intron13_500 bp upstream to | 22247-22790 | 22791-23404 | 23405-24080 |
| exon14_chr | | | |
| 1:68429928-1:68430427 | | | |
| 1:68448707-1:68449206 | 9742-9748 | 9784-9816 | 9818-9861 |
| Intron 1_500 bp Upstream to | 9750-9775 | 24984-25898 | 25899-26796 |
| Exon 2 | 9778-9781 | | |
| | 24081-24983 | | |
| 1:68449395-1:68449894 | 10307-10347 | 10351-10392 | 10393-10442 |
| Intron 1 | 26797-27377 | 27378-28025 | 28026-28727 |
| 1:68448124-1:68448623 | 9535-9536 | 9578-9616 | 9617-9658 |
| Intron 2_500 bp Downstream to | 9538-9541 | 29541-30385 | 30386-31251 |
| Exon 2 | 9543-9558 | | |
| | 9560-9561 | | |
| | 9563-9577 | | |
| | 28728-29540 | | |
| 1:68446861-1:68447360 | 8840-8885 | 8886-8933 | 8934-8983 |
| Intron 2_500 bp Upstream to | 9105-9109 | 9150-9154 | 9193-9197 |
| Exon 3 | 9111-9128 | 9156-9173 | 9199-9237 |
| | 9130-9135 | 9175-9191 | 32583-33248 |
| | 9137-9144 | 31917-32582 | |
| | 9146-9147 | | |
| | 31252-31916 | | |
| 1:68446210-1:68446709 | 8604-8648 | 8649-8693 | 8694-8740 |
| Intron 3_500 bp Downstream to | 33249-33901 | 33902-34570 | 34571-35257 |
| Exon 3 | | | |
| 1:68444884-1:68445383 | 8193-8200 | 8229-8260 | 8261-8298 |
| Intron 3_500 bp Upstream to | 8202-8211 | 36000-36777 | 36778-37575 |
| Exon 4 | 8214-8216 | | |
| | 8218-8223 8225 | | |
| | 8227-8228 | | |
| | 35258-35999 | | |
| 1:68444673-1:68444775 | 37576-37649 | 37650-37731 | 37732-37829 |
| Intron 4 | | | |
| 1:68444031-1:68444530 | 37830-38491 | 38492-39211 | 39212-39971 |
| Intron 5_500 bp Downstream to | | | |
| Exon 5 | | | |
| 1:68441001-1:68441500 | 39972-40665 | 40666-41395 | 41396-42151 |
| Intron 5_500 bp Upstream to | | | |
| Exon 6 | | | |
| 1:68440353-1:68440852 | 42152-42581 | 6501-6502 | 6504 6506-6510 |
| Intron 6_500 bp Downstream to | | 42582-43091 | 6512 6514 |
| Exon 6 | | | 43092-43657 |
| 1:68439643-1:68440142 | 6162-6180 | 6197-6212 | 6232-6248 |
| Intron 6_500 bp Upstream to | 6182-6189 | 6214-6224 | 6250-6251 |
| Exon 7 | 6191-6192 6194 | 6226-6227 6229 | 6253-6262 6264 |
| | 6196 43658- | 6231 44317- | 6266 45014- |
| | 44316 | 45013 | 45738 |
| 1:68439324-1:68439560 | 45739-46010 | 46011-46300 | 46301-46608 |
| Intron 7 | | | |
| 1:68439082-1:68439190 | 46609-46634 | 46635-46662 | 46663-46692 |
| Intron 8 | | | |
| 1:68438317-1:68438941 | 5416-5461 | 5462-5509 | 5510-5559 |
| Intron 9 | 5680-5722 | 5723-5766 | 5767-5814 |
| | 5920-5922 | 5965-5988 | 6000-6010 |
| | 5924-5930 5932 | 5990-5999 | 6012-6015 |
| | 5936 5938-5950 | 47602-48545 | 6017-6029 |
| | 5952 5955-5958 | | 6031-6041 |
| | 5961-5964 | | 48546-49516 |
| | 46693-47601 | | |

The indicated locations listed in column 1 of Table 1 are based on gnomAD v3 database and UCSC Genome Browser assembly ID: hg38, Sequencing/Assembly provider ID: Genome Reference Consortium Human GRCh38.p12 (GCA_000001405.27). Assembly date: December 2013 initial release; December 2017 patch release 12.
The SNP details are indicated by the listed SNP ID Nos. (“rs numbers”), which are based on the NCBI 2018 database of Single Nucleotide Polymorphisms (dbSNP)). The indicated DNA mutations are associated with Transcript Consequence NM 000329 as obtained from NCBI RefSeq genes.

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only.

### EXPERIMENTAL DETAILS

### Example 1: On-Target Screening Activity

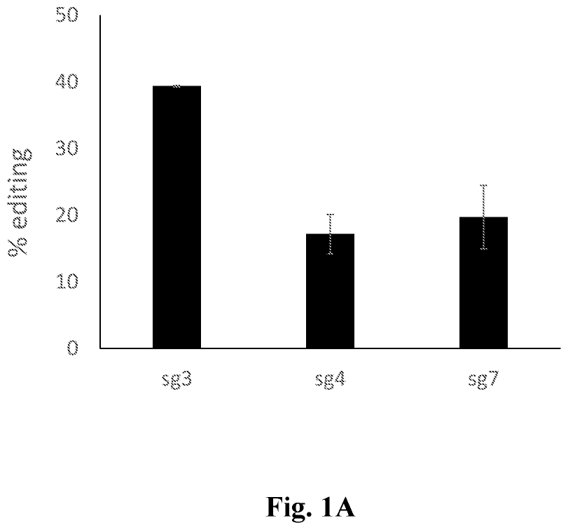

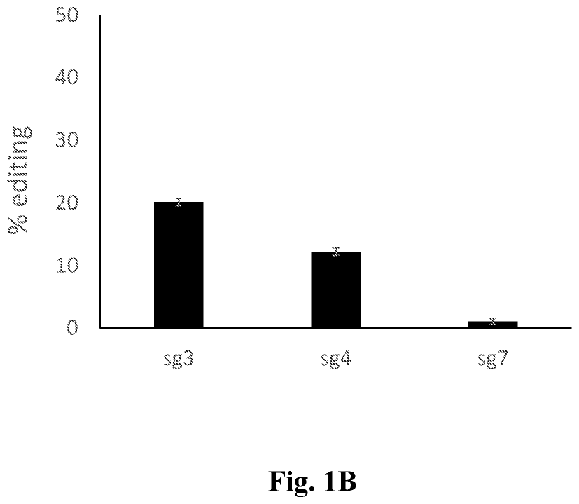

In order to choose optimal RNA guides for editing strategies of an RPE65 Asp477Gly mutation causing autosomal dominant retinitis pigmentosa, three different guides targeting the mutation were screened for high on-target activity in patient derived-iPSCs that harbor the pathogenic mutation. Briefly, 2.5×10⁵ iPSCs were mixed with pre-assembled RNPs composed of either (1) 105 pmole of SpCas9 protein and 120 pmole of 20 bp sgRNA or (2) 105 pmole of OMNI-50 protein and 120 pmole of 22 bp sgRNA. The sgRNAs target the mutated allele and are listed in Table 2. The RNP mix was combined with 100 pmole of electroporation enhancer (IDT-1075916) and electroporated using P3 Primary Cell 4D-nucleofector X Kit S (V4XP-3032, Lonza) by applying the CA-137 program. A fraction of cells were harvested 72 h post-nucleofection, genomic DNA was extracted, the region of the mutation was amplified, and the level of editing was analyzed by performing capillary electrophoreses. Edited amplicons, which contain indels, are distinguished from unedited amplicons according to their size. The graphs in FIG. 1A and FIG. 1B represent the average of % editing f STDV of two independent electroporation trials. According to capillary electrophoreses analysis, both SpCas9 (FIG. 1A) and OMNI-50 (FIG. 1B) guides displayed activity.

TABLE 2
SpCas9 and OMNI-50 sgRNA sequences targeting the
RPE65 mutation p.Asp477Gly (c.1430A > G).

| Guide name | Guide sequence | PAM |
|---|---|---|
| sg3_spCas | CAUCUUCUUCCAAGGCACCU (SEQ ID NO: 16) | GGG |
| sg4_spCas | UCAUCUUCUUCCAAGGCACC (SEQ ID NO: 34) | TGG |
| sg7_spCas | CCCAUCUUUGUUUCUCACCC (SEQ ID NO: 23) | AGG |
| sg3_OMNI50 | AUCAUCUUCUUCCAAGGCACCU (SEQ ID NO: | GGG |
| | 103) | |
| sg4_OMNI50 | CAUCAUCUUCUUCCAAGGCACC (SEQ ID NO: | TGG |
| | 111) | |
| sg7_OMNI50 | AACCCAUCUUUGUUUCUCACCC (SEQ ID NO: | AGG |
| | 95) | |

### REFERENCES

1. Ahmad and Allen (1992) “Antibody-mediated Specific Binging and Cytotoxicity of Lipsome-entrapped Doxorubicin to Lung Cancer Cells in Vitro”, Cancer Research 52:4817-20.
2. Anderson (1992) “Human gene therapy”, Science 256:808-13.
3. Basha et al. (2011) “Influence of Cationic Lipid Composition on Gene Silencing Properties of Lipid Nanoparticle Formulations of siRNA in Antigen-Presenting Cells”, Mol. Iher. 19(12):2186-200.
4. Behr (1994) Gene transfer with synthetic cationic amphiphiles: Prospects for gene therapy”, Bioconjuage Chem 5:382-89.
5. Blaese (1995) “Vectors in cancer therapy: how will they deliver”, Cancer Gene Ther. 2:291-97.
6. Blaese et al. (1995) “T lympocyte-directed gene therapy for ADA-SCID: initial trial results after 4 years”, Science 270(5235):475-80.
7. Browne et al. (2011) “A dominant mutation in RPE65 identified by whole-exome sequencing causes retinitis pigmentosa with choroidal involvement”, Eur. J. Hum. Genet. 19(10):1074-81.
8. Buchschacher and Panganiban (1992) “Human immunodeficiency virus vectors for inducible expression of foreign genes”, J. Virol. 66:2731-39.
9. Burstein et al. (2017) “New CRISPR-Cas systems from uncultivated microbes”, Nature 542:237-41.
10. Chang and Wilson (1987) “Modification of DNA ends can decrease end-joining relative to homologous recombination in mammalian cells”, Proc. Natl. Acad. Sci. USA 84:4959-4963.
11. Chung et al. (2006) “Agrobacterium is not alone: gene transfer to plants by viruses and other bacteria”, Trends Plant Sci. 11(1):1-4.
12. Coelho et al. (2013) “Safety and efficacy of RNAi therapy for transthyretin amyloidosis” N. Engl. J. Med. 369, 819-829.
13. Crystal (1995) “Transfer of genes to humans: early lessons and obstacles to success”, Science 270(5235):404-10.
14. Dillon (1993) “Regulation gene expression in gene therapy” Trends in Biotechnology 11(5):167-173.
15. Dranoff et al. (1997) “A phase I study of vaccination with autologous, irradiated melanoma cells engineered to secrete human granulocyte macrophage colony stimulating factor”, Hum. Gene Ther. 8(1):111-23.
16. Dunbar et al. (1995) “Retrovirally marked CD34-enriched peripheral blood and bone marrow cells contribute to long-term engraftment after autologous transplantation”, Blood 85:3048-57.
17. Ellem et al. (1997) “A case report: immune responses and clinical course of the first human use of ganulocyte/macrophage-colony-stimulating-factor-transduced autologous melanoma cells for immunotherapy”, Cancer Immunol Immunother 44:10-20.
18. Gao and Huang (1995) “Cationic liposome-mediated gene transfer” Gene Ther. 2(10):710-22.
19. Haddada et al. (1995) “Gene Therapy Using Adenovirus Vectors”, in: The Molecular Repertoire of Adenoviruses III: Biology and Pathogenesis, ed. Doerfler and Bohm, pp. 297-306.
20. Han et al. (1995) “Ligand-directed retro-viral targeting of human breast cancer cells”, Proc Natl Acad Sci U.S.A. 92(21):9747-51.
21. Inaba et al. (1992) “Generation of large numbers of dendritic cells from mouse bone marrow cultures supplemented with granulocyte/macrophage colony-stimulating factor”, J Exp Med. 176(6):1693-702.
22. Jinek et al. (2012) “A programmable dual-RNA-guided DNA endonuclease in adaptive bacterial immunity,” Science 337(6096):816-21.
23. Johan et al. (1992) “GLVR1, a receptor for gibbon ape leukemia virus, is homologous to a phosphate permease of Neurospora crassa and is expressed at high levels in the brain and thymus”, J Virol 66(3):1635-40.
24. Judge et al. (2006) “Design of noninflammatory synthetic siRNA mediating potent gene silencing in vivo”, Mol Ther. 13(3):494-505.
25. Kohn et al. (1995) “Engraftment of gene-modified umbilical cord blood cells in neonates with adnosine deaminase deficiency”, Nature Medicine 1:1017-23.
26. Kremer and Perricaudet (1995) “Adenovirus and adeno-associated virus mediated gene transfer”, Br. Med. Bull. 51(1):31-44.
27. Macdiarmid et al. (2009) “Sequential treatment of drug-resistant tumors with targeted minicells containing siRNA or a cytotoxic drug”, Nat Biotehcnol. 27(7):643-51.
28. Malech et al. (1997) “Prolonged production of NADPH oxidase-corrected granulocyes after gene therapy of chronic granulomatous disease”, PNAS 94(22):12133-38.
29. Miller et al. (1991) “Construction and properties of retrovirus packaging cells based on gibbon ape leukemia virus”, J Virol. 65(5):2220-24.
30. Miller (1992) “Human gene therapy comes of age”, Nature 357:455-60.
31. Mitani and Caskey (1993) “Delivering therapeutic genes—matching approach and application”, Trends in Biotechnology 11(5):162-66.
32. Nabel and Felgner (1993) “Direct gene transfer for immunotherapy and immunization”, Trends in Biotechnology 11(5):211-15.
33. Remy et al. (1994) “Gene Transfer with a Series of Lipphilic DNA-Binding Molecules”, Bioconjugate Chem. 5(6):647-54.
34. Sentmanat et al. (2018) “A Survey of Validation Strategies for CRISPR-Cas9 Editing”, Scientific Reports 8:888, doi:10.1038/s41598-018-19441-8.
35. Sharma et al. (2019) “Clinical-grade stem cell-derived retinal pigment epithelium patch rescues retinal degeneration in rodents and pigs.” Sci. Transl. Med. 11(475):eaat5580.
36. Sommerfelt et al. (1990) “Localization of the receptor gene for type D simian retroviruses on human chromosome 19”, J. Virol. 64(12):6214-20.
37. Van Brunt (1988) “Molecular framing: transgenic animals as bioactors” Biotechnology 6:1149-54.
38. Vigne et al. (1995) “Third-generation adenovectors for gene therapy”, Restorative Neurology and Neuroscience 8(1,2): 35-36.
39. Weissman and Kariko (2015) “mRNA:Fulfilling the promise of gene therapy”, Molecular Therapy (9):1416-7.
40. Wilson et al. (1989) “Formation of infectious hybrid virion with gibbon ape leukemia virus and human T-cell leukemia virus retroviral envelope glycoproteins and the gag and pol proteins of Moloney murine leukemia virus”, J. Virol. 63:2374-78.
41. Wright et al. (2013) “Complementation test of Rpe65 Knockout and tvrm148”, Invest Ophthalmol Vis Sci. 54(7):5111-22.
42. Yu et al. (1994) “Progress towards gene therapy for HIV infection”, Gene Ther. 1(1):13-26.
43. Zetsche et al. (2015) “Cpf1 is a single RNA-guided endonuclease of a class 2 CRIPSR-Cas system” Cell 163(3):759-71.
44. Zuris et al. (2015) “Cationic lipid-mediated delivery of proteins enables efficient protein based genome editing in vitro and in vivo” Nat Biotechnol. 33(1):73-80.

1. A method for modifying in a cell a mutant allele of the retinal pigment epithelium-specific 65 kDa protein (RPE65) gene having a mutation associated with a dominant RPE65 gene disorder, the method comprising
introducing to the cell a composition comprising:
at least one CRISPR nuclease or a sequence encoding a CRISPR nuclease; and
a first RNA molecule comprising a guide sequence portion having 17-25 nucleotides or a nucleotide sequence encoding the same,
wherein a complex of the CRISPR nuclease and the first RNA molecule affects a double strand break in the mutant allele of the RPE65 gene.

2. The method of claim 1, wherein the first RNA molecule targets the CRISPR nuclease to the mutation associated with a dominant RPE65 gene disorder, wherein the mutation associated with a dominant RPE65 gene disorder is any one of 1:68431085T>C and 1:68446825G>A (hg38), and
wherein the guide sequence portion of the first RNA molecule comprises 17-25 contiguous nucleotides containing nucleotides in the sequence set forth in any one of SEQ ID NOs: 1-49516 that targets a mutation associated with a dominant RPE65 gene disorder.

3. (canceled)

4. (canceled)

5. The method of claim 1, wherein the first RNA molecule targets the CRISPR nuclease to a SNP position of the mutant allele, wherein the SNP position is any one of rs60701104, rs9436400, rs868541802, rs3125890, rs75159457, rs1205919238, rs11209300, rs4264030, rs2419988, rs3118415, rs3118416, rs149739986, rs2182315, rs3118418, rs932783, rs12124063, rs77585943, rs1886906, rs3125891, rs11581095, rs12030710, rs1003041423, rs3118419, rs5774935, rs150459448, rs1555845, rs1555846, rs11269074, rs3790469, rs3125894, rs3125895, rs3125896, rs3125897, rs3125898, rs17130688, rs938759267, rs34194247, rs3125900, rs79716012, rs75711879, rs12145904, rs3125902, rs3118420, rs12138573, rs1925955, rs17130691, rs3125904, rs78507000, rs150774295, rs3125905, rs2038900, rs2038901, rs147665807, rs17130694, rs12408546, rs12077372, rs3790472, rs3790473, rs147893529, rs2012235, rs3118423, rs2986125, rs2986124, rs72926973, rs2277874, rs3125906, rs3118426, rs3125907, rs382422, rs3118427, rs3125908, rs12759602, rs2477974, rs3125909, rs3118428, rs12407140, rs72674322, rs72674323, rs3125910, and rs1318744874, and
wherein the guide sequence portion of the first RNA molecule comprises 17-25 contiguous nucleotides containing nucleotides in the sequence set forth in any one of SEQ ID NOs: 1-49516 that targets a SNP position of the mutant allele.

6. (canceled)

7. (canceled)

8. The method of claim 5, wherein the SNP position is in an exon or intron of the RPE65 mutant allele.

9. The method of claim 5, wherein the SNP position contains a heterozygous SNP.

10. The method of claim 1, further comprising introducing to the cell a second RNA molecule comprising a guide sequence portion having 17-25 nucleotides or a nucleotide sequence encoding the same, wherein a complex of the second RNA molecule and a CRISPR nuclease affects a second double strand break in the RPE65 gene.

11. The method of claim 10, wherein the guide sequence portion of the second RNA molecule comprises 17-25 contiguous nucleotides containing nucleotides in the sequence set forth in any one of SEQ ID NOs: 1-49516 other than the sequence of the first RNA molecule.

12. The method of claim 10, wherein the second RNA molecule comprises a non-discriminatory guide portion that targets both functional and mutated RPE65 alleles.

13. The method of a claim 10, wherein the second RNA molecule comprises a non-discriminatory guide portion that targets any one a region upstream to the RPE65 transcriptional start site, an intron of RPE65, and an intergenic region downstream of RPE65.

14. The method of claim 10, wherein the second RNA molecule comprises a non-discriminatory guide portion that targets a sequence that is located within a genomic range selected from any one of 1:68450655-1:68451154, 1:68428322-1:68428821, 1:68437687-1:68438186, 1:68431586-1:68432085, 1:68431377-1:68431469, 1:68431177-1:68431281, 1:68430565-1:68431064, 1:68429928-1:68430427, 1:68448707-1:68449206, 1:68449395-1:68449894, 1:68448124-1:68448623, 1:68446861-1:68447360, 1:68446210-1:68446709, 1:68444884-1:68445383, 1:68444673-1:68444775, 1:68444031-1:68444530, 1:68441001-1:68441500, 1:68440353-1:68440852, 1:68439643-1:68440142, 1:68439324-1:68439560, 1:68439082-1:68439190, and 1:68438317-1:68438941.

15. The method of claim 10, wherein the second RNA molecule comprises a non-discriminatory guide portion that targets a sequence that is located up to 500 base pairs from an exon that is excised by the first and second RNA molecules.

16. The method of claim 10, wherein an exon or a portion thereof is excised from the mutant allele of the RPE65 gene.

17. A modified cell obtained by the method of claim 1.

18. The method of claim 17, wherein the modified cell is a stem cell or a retinal pigment epithelium cell.

19. A first RNA molecule comprising a guide sequence portion having 17-25 contiguous nucleotides containing nucleotides in the sequence set forth in any one of SEQ ID NOs: 1-49516.

20. A composition comprising the first RNA molecule of claim 19 and at least one CRISPR nuclease.

21. The composition of claim 20, further comprising a second RNA molecule comprising a guide sequence portion having 17-25 contiguous nucleotides, wherein the second RNA molecule targets a RPE65 allele, and wherein the guide sequence portion of the second RNA molecule is a different sequence from the sequence of the guide sequence portion of the first RNA molecule.

22. The composition of claim 21, wherein the guide sequence portion of the second RNA molecule comprises 17-25 contiguous nucleotides containing nucleotides in the sequence set forth in any one of SEQ ID NOs: 1-49516 other than the sequence of the first RNA molecule.

23. A method for inactivating a mutant RPE65 allele in a cell, the method comprising delivering to the cell the composition of claim 20.

24. A method for treating a dominant RPE65 gene disorder, the method comprising delivering to a cell of a subject having a dominant RPE65 gene disorder the composition of claim 20.

25-28. (canceled)
